SEQUENCE LISTING (5 sequences)

SEQ ID NO: 1 - DNA, 2904 bp, Artificial Sequence
  Description of Artificial Sequence Synthetic polynucleotide
atggctagcgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggtggaggaatggtgagcgagctgattaaggagaacatgcccatgaagctgtacatggagggcaccgtgaacaaccaccacttcaagtgcacatccgagggcgaaggcaagccctacgagggcacccagaccatgagaatcaaggtcgtcgagggcggccctctccccttcgccttcgacatcctggctaccagcttcatgtacggcagcagaaccttcatcaagcaccctccgggcatccccgacttctttaagcagtccttccctgagggcttcacatgggagagagtcaccacatacgaagacgggggcgtgctgaccgctacccaggacaccagcctccaggacggctgcctcatctacaacgtcaaggttagaggggtgaacttcccagccaacggccctgtgatgcagaagaaaacactcggctgggaggcctccaccgagacgatgtaccccgctgacggcggcctggaaggcgcatgtgacatggccctgaagctcgtgggcgggggccacctgatctgcaaccttgagaccacatacagatccaagaaacccgctacgaacctcaagatgcccggcgtctacaacgtggaccacagactggaaagaatcaaggaggccgacgatgagacctacgtcgagcagcacgaggtggctgtggccagatactctactggtggcgctggtgatggaggtaaagcggccacgcgtatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcagcccgggatccaccggtcgccaccggttcagggagcggatccggc

SEQ ID NO: 2 - DNA, 2904 bp, Artificial Sequence
  Description of Artificial Sequence Synthetic polynucleotide
atggctagcgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggtggaggaatggtgagcgagctgattaaggagaacatgcccatgaagctgtacatggagggcaccgtgaacaaccaccacttcaagtgcacatccgagggcgaaggcaagccctacgagggcacccagaccatgagaatcaaggtcgtcgagggcggccctctccccttcgccttcgacatcctggctaccagcttcatgtacggcagcagaaccttcatcaagcaccctccgggcatccccgacttctttaagcagtccttccctgagggcttcacatgggagagagtcaccacatacgaagacgggggcgtgctgaccgctacccaggacaccagcctccaggacggctgcctcatctacaacgtcaaggttagaggggtgaacttcccagccaacggccctgtgatgcagaagaaaacactcggctgggaggcctccaccgagacgatgtaccccgctgacggcggcctggaaggcgcatgtgacatggccctgaagctcgtgggcgggggccacctgatctgcaaccttgagaccacatacagatccaagaaacccgctacgaacctcaagatgcccggcgtctacaacgtggaccacagactggaaagaatcaaggaggccgacgatgagacctacgtcgagcagcacgaggtggctgtggccagatactctactggtggcgctggtgatggaggtaaagcggccacgcgtatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtggagcacagatcatgatcggagcagcagcccgggatccaccggtcgccaccggttcagggagcggatccggc

SEQ ID NO: 3 - DNA, 2523 bp, Artificial Sequence
  Description of Artificial Sequence Synthetic polynucleotide
atggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctatcctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggtggaggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggcggccacgcgtatgagcaattcagttttgtccacttcttccccaggtaatatccaaactcggcaggcgattccggcccaactcctctattccctcgtcaaccaaaaagccagcggcaaacttgtggtacaaaacccctttgacgaatttgtttattggcaagtttatttgggggatggtcgtattcacttggccaacagcgccaatggttgttccgaaagacttaattatctgattggtagcacccttcgtcagaagggcattgaactgccatcaagaattgctgacgattatatttttctttccgggctctggaaaagggatgttttttcctttcagcaaacccgctcgattctgacccaagctagccaagaagccttagttcaggtactttccttacctaaaacatcctatcgttttaatcaagaaaaagaactgagcgaactatttttgaatttagagttagaaaaaactattacccccatcaaacataaaattcgctattggtgggaattgaggtcctatattaactcgcctttccagcgacccctagtggaaaactggcttagtctgcgccaaaatttggtgaaaaataacctccatggccaacattggctaaaacgcttccacacttgcttggaaaatctaaattgcttatatgaaattgccgggcatttggaaatcagtactttggagttggcattaatgctacagcccatggtcaagactggcgaaatcaagatgctgccttatcaagaaattcaaacggatgaccgccctttggtggcttatgttgaccatagggaagtccagcagcgcatgatgacctattctttggaaaaagaaggctatcgagccttagtactggatgatccctttcgggctttaacggtgttgttgaattctccccccaatctgattatcataaatactgacttgccggacatgaacggttatcaactctgttcgttttgccgtaagtccaatcaacttaaagatgtgccgatcctactattggggaaaggtgacaatttgatcagcggtattcgtatcaaactttgtcaagcttctgggtacttaagtgagccttttgttccagaagaattactcgatcgggtggcaaaactccttcccaccaataaaaccaactcctga

SEQ ID NO: 4 - DNA, 1449 bp, Artificial Sequence
  Description of Artificial Sequence Synthetic polynucleotide
atggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagggtggaggaatggtgagcgagctgattaaggagaacatgcccatgaagctgtacatggagggcaccgtgaacaaccaccacttcaagtgcacatccgagggcgaaggcaagccctacgagggcacccagaccatgagaatcaaggtcgtcgagggcggccctctccccttcgccttcgacatcctggctaccagcttcatgtacggcagcagaaccttcatcaagcaccctccgggcatccccgacttctttaagcagtccttccctgagggcttcacatgggagagagtcaccacatacgaagacgggggcgtgctgaccgctacccaggacaccagcctccaggacggctgcctcatctacaacgtcaaggttagaggggtgaacttcccagccaacggccctgtgatgcagaagaaaacactcggctgggaggcctccaccgagacgatgtaccccgctgacggcggcctggaaggcgcatgtgacatggccctgaagctcgtgggcgggggccacctgatctgcaaccttgagaccacatacagatccaagaaacccgctacgaacctcaagatgcccggcgtctacaacgtggaccacagactggaaagaatcaaggaggccgacgatgagacctacgtcgagcagcacgaggtggctgtggccagatactctactggtggcgctggtgatggaggtaaagcggccacgcgtatgagtttgtaccgtttgatttacagcagtcagggcattcccaatttacaaccccaggatttaaaagatatcttagaatcttcccaaagaaataatccggccaatggcattaccggactgctctgctacagcaagccggcttttctgcaggtattggaaggagagtgtgaacaggttaatgaaacctaccaccggattgtgcaagatgaacgccaccacagcccccagatcattgagtgtatgccgatcagaagaagaaacttcgaggtttggtctatgcaagcgatcacggtgaatgatttgtcaacggaacaggttaagactttggtattgaaatattccggcttcaccaccctacggcccagtgccatggacccggaacaatgcctaaactttttgctagacattgccaagatttacgagttgtccgataactttttcctcgacctc

SEQ ID NO: 5 - DNA, 1449 bp, Artificial Sequence; identical to SEQ ID NO: 4
  Description of Artificial Sequence Synthetic polynucleotide

(19) United States
(12) Patent Application Publication
(10) Pub. No.: US 2021/0062165 A1
(43) Pub. Date: Mar. 4, 2021

(54) SYSTEM AND METHOD OF INDUCIBLY CLUSTERING METABOLIC ENZYMES FOR THE PRODUCTION OF CHEMICALS USING CELL FACTORIES

(71) Applicant: The Trustees of Princeton University, Princeton, NJ (US)

(72) Inventors: Jose L. Avalos, Princeton, NJ (US); Jared E. Toettcher, Princeton, NJ (US); Clifford P. Brangwynne, Hopewell, NJ (US); Evan M. Zhao, Clarence Center, NY (US); Maxwell Z. Wilson, Princeton, NJ (US)

(73) Assignee: The Trustees of Princeton University, Princeton, NJ (US)

(21) Appl. No.: 17/003,659

(22) Filed: Aug. 26, 2020

Related U.S. Application Data
(60) Provisional application No. 62/891,762, filed on Aug. 26, 2019.

(51) Int. Cl. C12N 9/02; C12N 13/00; C12N 15/81

(52) U.S. Cl. CPC C12N 9/0073; C07K 2319/60; C12N 15/81; C12N 13/00

(57) ABSTRACT

Provided herein is a system and method of optogenetically inducibly clustering metabolic enzymes for the production of chemicals using cell factories. More particularly, the described inducible protein clustering approach clusters metabolic enzymes by, e.g., a change in illumination conditions (either a switch from dark to light or from light to dark). Performing this clustering leads to an increase in the production of metabolites by the clustered enzymes. In some embodiments, a light-sensitive domain may be replaced with any inducible domain.

### CROSS-REFERENCE TO RELATED APPLICATIONS

This application claims the benefit of U.S. Provisional Application No. 62/891,762, filed on Aug. 26, 2019 which is herein incorporated by reference in its entirety.

### STATEMENT REGARDING FEDERALLY SPONSORED RESEARCH OR DEVELOPMENT

This invention was made with government support under Grant Nos. EB024247 and DA040601 awarded by the National Institutes of Health. The government has certain rights in the invention.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Aug. 26, 2020, is named Princeton-63502_SL.txt and is 16,015 bytes in size.

### BACKGROUND

Cellular metabolism is an intricate web of biochemical reactions. This network relies on the same intermediates to satisfy many different nutrient and energy synthesis requirements. The fluxes of the various branches require temporal regulation, which is especially important for the engineering of metabolism for chemical production. Engineers must balance flux towards essential metabolic products for maintaining organism viability and flux towards desired products for industrial applications.

Current techniques vary enzyme levels to achieve this balance but are constrained by the energy costs of protein degradation and timescales of enzyme expression. In contrast, endogenous regulation of metabolism additionally utilizes fast, reversible perturbations, By managing existing enzymes through post-translational modifications, such as phosphorylation, compartmentalization into organelles, or reversible enzymatic clustering, cells can rapidly switch in response to diverse signals without changing total enzyme quantities.

Metabolic engineers have employed organelle: localization of metabolic pathways of interest to enhance microbial chemical production, but are limited by lack of dynamic control, enzyme and metabolite transport, and competition with endogenous intra-organelle enzymes.

Current techniques have yet to capture the full benefits of the dynamic compartmentalization found in nature. An ideal system for metabolic perturbations would enable on-demand shunting of flux down a pathway of interest by dynamically controlling enzyme activity or localization independently of enzyme levels. This system could be also crucial for the overproduction of toxic products that interfere with normal cell function.

Clustering and compartmentalization work by increasing local enzyme concentrations in a pathway of interest to accelerate intermediate processing and sequester toxic intermediates. Colocalizing two metabolic enzymes that operate at sequential steps enables the product of the first enzyme to encounter the second before it diffuses away, thereby increasing the local concentration of substrate for the second enzyme and enhancing flux. This phenomenon is important for the regulation of metabolism at multiple stages, including glycolysis regulation and purine biosynthesis. Purine biosynthesis enzymes form phase-separated membraneless organelles upon purine starvation with reversible dynamics.

Cellular function relies on coordinating the thousands of reactions that simultaneously take place within the cell. Cells accomplish this task in large part by spatio-temporally controlling these reactions using diverse intracellular organelles. In addition to classic membrane-bound organelles such as secretory vesicles, mitochondria and the endoplasmic reticulum, cells harbor a variety of membraneless organelles. From a biophysical standpoint, these structures are remarkable in that they have no enclosing membrane and yet their overall size and shape may be stable over long periods (hours or longer), even while their constituent molecules exhibit dynamic exchange over timescales of tens of seconds (Phair and Misteli, 2000). Moreover, many of these structures have recently been shown to exhibit additional behaviors typical of condensed liquid phases. For example, P granules, nucleoli, and a number of other membraneless bodies will fuse into a single larger sphere when brought into contact with one another ((Brangwynne et al., 2009), (Brangwynne et al., 2011), (Feric and Brangwynne, 2013)), in addition to wetting surfaces and dripping in response to shear stresses. These observations have led to the hypothesis that membraneless organelles represent condensed liquid states of RNA and protein that assemble through intracellular phase separation, analogous to the phase transitions of purified proteins long observed in vitro by structural biologists ((Ishimoto and Tanaka, 1977), (Vekilov, 2010)). Consistent with this view, ribonucleoprotein (RNP) bodies or granules, and other membraneless organelles appear to form in a concentration-dependent manner, as expected for liquid-liquid phase separation ((Brangwynne et al., 2009), (Weber and Brangwynne, 2015), (Nott et al., 2015), (Wippich et al., 2013), (Molliex et al., 2015)).

Weak multivalent interactions between molecules containing tandem repeat protein domains appear to play a central role in the molecular driving forces and biophysical nature of intracellular phases ((Li et al., 2012), (Banjade and Rosen, 2014)). A related driving force is the interaction (e.g. electrostatic, dipole-dipole) between segments of conformationally heterogeneous proteins, known as intrinsically disordered protein or intrinsically disordered regions (IDP/IDR, which are typically, although not necessarily, also low complexity sequences, LCS). Hereinafter, the terms intrinsically disordered protein, intrinsically disordered region, and intrinsically disordered protein region are used interchangeably. RNA binding proteins often contain IDRs with the sequence composition biased toward amino acids including R, G, S, and Y, which comprise sequences that have been shown to be necessary and sufficient for driving condensation into liquid-like protein droplets ((Elbaum-Garfinkle et al., 2015), (Nott et al., 2015), (Lin et al., 2015)). The properties of such in vitro droplets have recently been found to be malleable and time-dependent ((Elbaum-Garfinkle et al., 2015), (Zhang et al., 2015), (Weber and Brangwynne, 2012), (Molliex et al., 2015), (Lin et al., 2015), (Xiang et al., 2015), (Patel et al., 2015)), underscoring the role of IDR/LCSs in both liquid-like physiological assemblies and pathological protein aggregates.

There is a need for an approach to assemble or disassemble synthetic structures on demand to enable inducible control over metabolic flux in engineered metabolic pathways for efficient, reversible regulation of small molecule biosynthesis. Such an approach would be highly desirable.

### SUMMARY

There is a need for inducible control over metabolic flux to control engineered metabolic pathways.

The present invention employs synthetic organelles for dynamic, reversible control over enzyme localization and metabolic flux for efficient and reversible regulation of small molecule. biosynthesis. Described herein are methods for reliable optogenetic assembly and disassembly of synthetic organelle formation in cells such as microbes and eukaryotic cells, for example, yeast. Light-dependent formation of these organelles can enhance metabolic flux by colocalizing enzymes that operate at sequential steps. Using the violacein pathway as a model system, we demonstrate that synthetic compartmentalization of enzymes at a metabolic branch point increases flux towards one branch and show a 6.1+/−0.9 fold-change for a two-enzyme pathway, achieving the theoretical maximum expected fold-change for two-enzyme colocalization.

Recent advances in inducible phase separation with light-activated proteins provides a unique opportunity to manipulate metabolic pathways using synthetic organelles. We demonstrate that two different synthetic organelle formation systems can lead to a fold-change in target product titer. Our work demonstrates how light-induced synthetic organelles can be harnessed to increase specificity of a metabolic pathway.

Nevertheless, this technique comes with pathway-dependent challenges. Reversible clustering activity occurs only within a narrow range of enzyme concentration and can differ between enzymes. Even within the same metabolic pathway, expression levels that work for one enzyme may not work for another, as seen with VioEp described herein. Multimer forming enzymes can constitutively activate Fused in Sarcoma (FUS)-activated organelle formation. We have partially addressed this issue through the development of a systematic integration method to sample a wide range of expression levels. However, one parameter we did not address was the enzymatic density within these synthetic organelles, something that could lead to even greater changes in flux shuttling that may be used for the adaptation of this technique to other pathways. This parameter can be tuned by also including the clustering tags without enzyme fusions into the phase separated organelles, thus increasing the amount of “free space” within the separation.

Another challenge to using this technique is the compatibility of enzymes with their clustering tags. We employed the violacein system due to the availability of crystal structures which predicted tolerance to large protein tags. Clustering tags can be fused at the N- or C-terminus tags or smaller clustering tags can be chosen.

Selection of different concentrations of clustering tag-enzyme fusions can address variation in [Zeo]ₘₐₓ (the highest level of zeocin) required for light-dependent synthetic organelle formation.

The utility of synthetic organelles increases with pathway complexity. Theoretically, the maximum flux change through a 2 enzyme complex is 6-fold while a 3 enzyme complex is 110-fold. These changes become more relevant for the low-yield products of complicated pathways such as those involved in vital pharmaceutical targets. Furthermore, complex metabolic pathways often contain toxic intermediates or branches which result in toxic products. For example, in the production of artemisinin, an antimalarial drug, it is vital to prevent accumulation of the toxic intermediate, isopentenyl pyrophosphate. in these difficult pathways, the Cry2Drop and PIXELL systems can manipulate flux away from the toxic product with a light input. Toxic intermediate enzymes that are required for final product synthesis may be dispersed when the cells can grow to an optimal density and recruited to a different synthesis organelle for product production.

Described herein are new methods for inducible compartmentalization in synthetic organelles that can be used in metabolic manipulation. Through the violacein pathway, we demonstrate that the proposed system works in complicated enzymatic systems through light-dependent shuttling of flux. Furthermore, we not only show that this method is compatible with two different light-dependent phase separation systems, but also that both systems can be used in parallel. These new methods offer new opportunities in metabolic engineering, the study of metabolism, and small molecule biosynthesis.

In one embodiment, optogenetics is used to control protein interactions to maximize a product of interest in engineered metabolic pathways.

In another embodiment, the optogenetic system is a Cry2 system.

In another embodiment, the optogenetic system is a Cry2olig system.

In another embodiment, the optogenetic system is a PixELL system

Another embodiment is a synthetic protein, comprising at least one inducible domain attached to an open reading frame (ORF) of a metabolic enzyme, wherein the inducible domain changes conformation in a manner that leads to oligomerization of the protein.

Another embodiment is a method of controlling metabolic flux in a synthetic organism, comprising the steps of providing a synthetic organism and controlling the metabolic flux by exposing the synthetic organism to a first condition at a first point in time.

In some embodiments, the synthetic organism is yeast, bacteria, mold, alga, plant, or a mammalian cell.

### BRIEF DESCRIPTION OF THE SEQUENCES

SEQ ID NO:1 is the gene sequence encoding FUS^{N}-FusionRed-Cry2 fusion protein.

SEQ ID NO: 2 is the gene sequence encoding FUS^{N}-FusionRed-Cry2olig fusion protein.

SEQ ID NO:3 is the gene sequence encoding FUS^{N}-Citrine-PixE fusion protein.

SEQ ID NO:4 is the gene sequence encoding FUS^{N}-FusionRed-PixD fusion protein.

SEQ ID NO:5 is the gene sequence encoding sFUS-FusionRed-PixD fusion protein.

### BRIEF DESCRIPTION OF THE DRAWINGS

The patent or application file contains at least one drawing executed in color.

Copies of this patent or patent application publication with color drawings will be provided by the Office upon request and payment of the necessary fee.

FIGS. 1A-1C are schematic diagrams of metabolic flux between different branches in a pathway. FIG. 1A illustrates metabolites A, B, C, and D are converted through the action of enzymes E₁, E₂ and E₃, FIG. 1B illustrates, under conditions where enzymes E₂ and E₃ are in competition, metabolic flux of metabolite B will be split between final products C and D. FIG. 1C shows if one branch is localized into a separated organelle including E₁ and E₃, metabolite flux can be shunted toward the product of interest, D, thereby reducing production of unwanted product.

FIGS. 1D and 1E illustrate two methods for optogenetic regulation of membraneless organelle assembly. FIG. 1D shows FUS^{N} fusions to variants of the Cry2 optogenetic system lead to light-induced protein phase separation in the optoDroplet and optoCluster systems. FIG E shows FUS^{N} fusions to PixD/E proteins form the light-dissociable PixELL optogenetic system.

FIG. 2A-2D illustrate redirecting flux in the deoxyviolacein pathway using light-inducible optoClusters. FIG. 2A shows light induced co-clustering of VioE and VioC enzymes increases VioC induced production of deoxyviolacein (DV) and limits production of prodeoxyviolacein (PDV). FIG. 2B shows optoCluster constructs. FIG. 2C is a graph showing high-performance liquid chromatography (HPLC) quantification of PDV from yeast colonies with [Zeo]ₘₐₓ=800 mgl⁻¹ strains YNS34 (optoCluster-VioC and optoCluster-VioE) and YNS36 (VioC-optoCluster and optoCluster-VioE) incubated in dark or blue light, and FIG. 2D is a graph showing HPLC quantification of DV from yeast colonies with [Zeo]ₘₐₓ=800 mgl⁻¹ strains YNS34 (optoCluster-VioC and optoCluster-VioE) and YNS36 VioC-optoCluster and optoCluster-VioE) incubated in dark or blue light.

FIG. 2E is a graph showing HPLC quantification of PDV in four yeast strains (shown in graph bars from left to right: YNS54, YNS55, YNS56, YNS57) lacking co-clustered VioC and VioE, incubated in dark or blue light.

FIG. 2F is a microscopy image of strain YNS34 with [Zeo]ₘₐₓ=800 mgl⁻¹, under dark and blue-light conditions.

FIG. 3A illustrates redirecting flux in the PDV pathway using light-disscociable PixELLs. Co-clustering VioE and VioC enzymes in the dark increases VioC-induced production of DV and limits production of PDV. PixELLs are dissociated in light resulting in loss of enhanced DV production.

FIG. 3B shows PixELL constructs tested for darkness-induced deoxyviolacein production.

FIGS. 3C-E are graphs showing YEZ257 colony with [Zeo]ₘₐₓ=1,200 mg l⁻¹ HPLC quantification of PDV (FIG. 3C), DV (FIG. 3D), and DV/PDV ratio (FIG. 3E) in the dark and light using PixELLs. Scale bar, 5μ. Error bars represent s.d. of four 1-ml biological replicates.

FIG. 3F is a graph showing HPLC quantification of PDV for four day fermentations of strains YEZ281 and YEZ512 lacking co-clustered VioE and VioC.

FIG. 3G shows microscopy images of YEZ257 under different light conditions showing constitutively clustered VioE but light-induced delocalization of VioC.

FIG. 4A is a schematic diagram of light switchable metabolic flux control at an enzymatic branch point. Co-clustering VioE and VioC enhances flux of DV production, and suppresses an alternative VioD-catalyzed branch that produces proviolacein and violacein. PixELLs are dissociated in the light. This illustration depicts co-clustering enhancement of DV in the dark and loss of DV enhancement on blue light stimulation.

FIG. 4B is a graph showing HPLC quantification of proviolacein, violacein, DV and PDV from four day fermentations of strain YEZ511. Error bars represent s.d. of four 1 ml biological replicates (shown as individual points). *P<0.05, **P<0.01, ***P<0.001. Statistics are derived using a one-sided t-test.

FIG. 5 depicts the violacein synthesis pathway.

FIG. 6 is a general vector map showing the relative orientation of the three positions listed in Table 1 in which different genes (including promoters and terminators) were assembled, using a multiple gene insertion strategy. The vectors have an ampicillin resistance marker (AMPR) for cloning in E. coli and a selection marker for S. cerevisiae (Marker). Vector types include CEN/ARS, 2μ or integrative.

FIG. 7 shows IDR shortening improves enzyme activity and/or expression. Investigation of PixELLs functionality using constructs with the first 93 amino acids of the FUS^{N} domain. FIG. 7A shows microscopy images of YEZ555 under different light conditions showing dissociation of the two components of shortened PixELL system. Images are representative of four colonies picked at the conditions specified. FIG. 7B shows constructs tested for dark-inducible metabolic organelles harboring shortened FUS domains (sFUS) on PixELLs-based VioE/VioC co-clusters. FIG. 7C shows HPLC quantification of PDV from four day fermentations of YEZ553 (left) and YEZ554 (right, control with no VioC), both of which were selected from [Zeo]ₘₐₓ=1,200 mg/L. Error bars represent standard deviations of 1 mL biological replicates exposed to the same light conditions (n=4).***, p<0.001. Statistics are derived using a one-sided t test. FIG. 7D shows HPLC quantification of DV from four day fermentations of YEZ553 (left, shortened PixELLs) and YEZ257 (right, Full-length PixELLs), both of which were selected from [Zeo]ₘₐₓ=1,200 mg/L. All data are shown as mean values; dots represent individual data points; error bars represent the standard deviation of four biologically independent 1-ml sample replicates exposed to the same conditions.

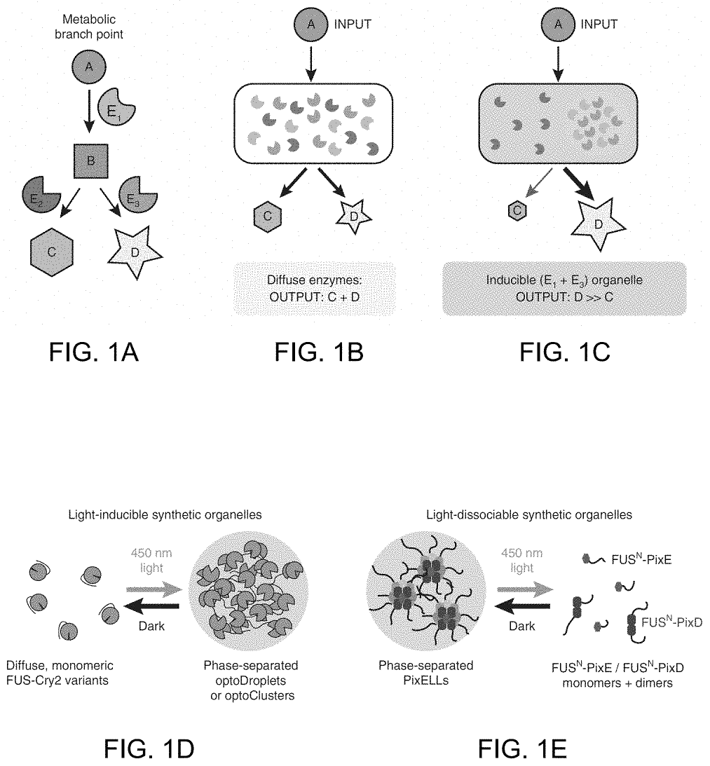

### DETAILED DESCRIPTION

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a”, “an”, and “the” include plural referents unless the context clearly dictates otherwise.

Described herein is a system and method of inducibly clustering metabolic enzymes for the production of chemicals using cell factories. More particularly, the light-induced protein clustering approach described herein clusters metabolic enzymes by a change in illumination conditions (either a switch from dark to light or from light to dark). Performing this clustering leads to an increase in the production of metabolites by the clustered enzymes. Similarly, sometimes the un-clustering of enzymes results in an increase of a metabolite. These increases in metabolite production are the result of shifting the metabolic flux from one path to another. In some embodiments, a light-sensitive domain may be replaced with any inducible domain.

The present invention can be employed to dramatically increase the production of desired chemicals by metabolic enzymes in living cells. Proof-of-concept experimental results have shown that clustering works on one pair of enzymes. The approach described herein can also be applied to the production of biofuels and other valuable products such as, for example, small molecule biosynthesis and pharmaceutical production. It is expected that the described approach and related “optogenetic” techniques will dramatically change the way that metabolic engineers control flux through engineered pathways.

Currently, the metabolic engineering industry lacks an effective way of controlling protein activity directly. The described technology allows for easy, tunable control of metabolic flux at the protein level (post-translationally). Being able to shift enzyme activity on or off, (reversibly and repeatedly) at different time points allows for protein level optimization of microbial chemical production processes that is impossible with other techniques. This will help improve the chemical titers these microbial organisms can output.

The described approach is a process by which light-inducible clustering tags are attached onto metabolic enzymes to direct flux with light activation and is done by attaching a clustering tag to the open reading frame (ORF) of a metabolic enzyme through plasmid construction or genetic alteration. The clustering is then activated through blue light (typically wavelengths from about 450 nm to about 495 nm) stimulation.

Also described herein are compositions of matter, engineered proteins where a switch from light to darkness or darkness to light changes the clustering state.

In some embodiments, a light-sensitive domain may be replaced with any inducible domain. When the inducible domain is able to form quaternary structure, the induction of the quaternary complex can drive phase separation of protein clusters. Typically, this will involve intrinsically disordered domains, although other domains may be utilized (see, e.g., Shin et al. 2017, Cell, below). Examples of induction methods that could lead to this effect are pH, nutrients (e.g., purine starvation will lead to purinosome formation), metal (e.g., cadmium binding complexes and ferritin), chemicals and protein agents (e.g., beta-estradiol).

While clustering tags can affect protein activity and negatively impact the flux through the desired pathway, picking enzymes that can tolerate larger tags can ameliorate this problem, as would engineering new proteins with smaller light-induced clustering domain.

A proof of concept yeast strain was constructed that demonstrates the capability of clustering for the upregulation of deoxyviolacein under blue light. This approach can be translated to any pair or group of enzymes that tolerates clustering tags. Similarly, another strain was constructed that demonstrates the capability of clustering that is inducible by placing the strain in darkness.

### REFERENCES

Shin, Yongdae, et al. “Spatiotemporal control of intracellular phase transitions using light-activated optoDroplets”. Cell 168, 159-171, Jan. 12, 2017.

United States Patent Application Publication 2017/0355977 A1, Brangwynne et al., Optogenetic Tool For Rapid And Reversible Clustering Of Proteins, Dec. 14, 2017.

Zhao, Evan, et al., Light-based control of metabolic flux through assembly of synthetic organelles, Nature Chemical Biology 15(6) 589-597 (2019).

The references listed herein are incorporated by reference in their entirety as if fully set forth herein.

Optogenetic systems, for example, the Cry2, Cry2olig, optoDroplets, optoClusters and/or PixELL systems, may be employed for the approaches described herein. The Cry2 droplet system is described in U.S. Patent Application Publication 2017/0355977 A1 incorporated herein by reference. Cry2 oligomerization may be enhanced by a point mutation to provide Cry2olig. Fusion proteins FUS^{N}-Cry2 and FUS^{N}-Cry2olig were created by fusion of Cry2 and Cry2olig to the N terminal intrinsically disordered region (IDR) from the protein FUS (i.e., FUS^{N}).

Fusion proteins or synthetic proteins for use in the systems and methods described herein to inducibly cluster metabolic enzymes for the production of desired chemicals include, but are not limited to, the FUS intrinsically-disordered region (FUS^{N}), a fluorescent protein domain and an optogenetic system protein domain. Examples of such fusion proteins include FUS^{N}-FusionRed-Cry2, FUS^{N}-FusionRed-Cry2olig, FUS^{N}-Citrine-PixE, FUSN-FusionRed-PixD, and sFUS-FusionRed-PixD. DNA sequences encoding these fusion proteins are provided in SEQ ID NOS. 1-5 of the sequence listing:

SEQ ID NO:1 is the gene sequence encoding FUS^{N}-FusionRed-Cry2 fusion protein.

SEQ ID NO: 2 is the gene sequence encoding FUS^{N}-FusionRed-Cry2olig fusion protein.

SEQ ID NO:3 is the gene sequence encoding FUS^{N}-Citrine-PixE fusion protein.

SEQ ID NO:4 is the gene sequence encoding FUS^{N}-FusionRed-PixD fusion protein.

SEQ ID NO:5 is the gene sequence encoding sFUS-FusionRed-PixD fusion protein.

FUS^{N}-Cry2 forms liquid-like spherical droplets that rapidly exchange monomers in and out of clusters referred to herein as optoDroplets. FUS^{N}-Cry2olig, referred to herein as optoClusters, form rigid clusters that do not exchange subunits with the solution.

The PixELL system has an inverted light response. FUS^{N} fusions to the PixD/E proteins form the light dissociable PixELL optogenetic system. When co-expressed in cells, FUS^{N}PixD and FUS^{N}PixE form liquid-like droplets which disassemble when exposed to blue light illumination, for example, 450 nm blue light illumination.

In an embodiment, the fusion protein comprises a light-responsive domain and a heterologous peptide component, wherein exposure of the fusion protein to light induces a conformational change in the fusion protein that alters an activity of the fusion protein.

The term “fusion protein” refers to a synthetic, semi-synthetic or recombinant single protein molecule that comprises all or a portion of two or more different proteins and/or peptides. The fusion can be an N-terminal fusion (with respect to the heterologous peptide component), a C-terminal fusion (with respect to the heterologous peptide component) or an internal fusion (with respect to the light responsive domain and/or the heterologous peptide component). When the fusion protein is an internal fusion protein, the light responsive domain is typically inserted into the heterologous peptide component. Thus, in some embodiments, the fusion protein is an internal fusion protein, and the light responsive domain (e.g., LOV domain) is inserted into the heterologous peptide component.

Fusion proteins of the invention can be produced recombinantly or synthetically, using routine methods and reagents that are well known in the art. For example, a fusion protein of the invention can be produced recombinantly in a suitable host cell (e.g., bacteria, yeast, insect cells, mammalian cells) according to methods known in the art. See, e.g., Current Protocols in Molecular Biology, Second Edition, Ausubel et al. eds., John Wiley & Sons, 1992; and Molecular Cloning: a Laboratory Manual, 2nd edition, Sambrook et al., 1989, Cold Spring Harbor Laboratory Press. For example, a nucleic acid molecule comprising a nucleotide sequence encoding a fusion protein described herein can be introduced and expressed in suitable host cell (e.g., E. coli), and the expressed fusion protein can be isolated/purified from the host cell (e.g., in inclusion bodies) using routine methods and readily available reagents. For example, DNA fragments coding for different protein sequences (e.g., a light-responsive domain and a heterologous peptide component) can be ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992).

The fusion proteins described herein can include other amino acid sequences in addition to the amino acid sequences of the light-responsive domain and the heterologous peptide component. In some aspects, a fusion protein includes a linker amino acid sequence (e.g., positioned between the light-responsive domain and the heterologous peptide component). A variety of linker amino acid sequences are known in the art and can be used in the fusion proteins described herein. In some embodiments, a linker sequence includes one or more amino acid residues selected from Gly, Ser, Ala, Val, Leu, Ile, Thr, His, Asp, Glu, Asn, Gln, Lys and Arg. In some embodiments, a linker sequence includes a polyglycine sequence (e.g., a 6X glycine sequence).

In some aspects, the fusion protein is isolated. As used herein, “isolated” means substantially pure. For example, an isolated fusion protein makes up at least about 50%, about 60%, about 70%, about 80%, about 90%, about 95%, about 97%, about 98%, about 99% or about 99.5% by weight of a mixture containing substances (e.g., chemicals, proteins, peptides, other biological matter) other than the fusion protein.

“Light-responsive domain,” as used herein, refers to a peptide or protein that, upon exposure to at least one particular wavelength of light (more typically, a range of wavelengths of light), undergoes a conformational change which mediates, in turn, a conformational change in the fusion protein. Conformational changes include unfolding, tilting, rotating and multimerizing (e.g., dimerizing, trimerizing), or a combination of any of the foregoing (e.g., unfolding and multimerizing). Accordingly, in some aspects, the conformational change is an allosteric change, such as the allosteric change undergone by AsLOV2 upon exposure to blue light. In some aspects, the conformational change induces multimerization (e.g., dimerization, trimerization) of the fusion protein.

Typically, the light-responsive domain is an optogenetic activator from plants, fungi, or bacteria. Non-limiting examples of light responsive domains include light oxygen voltage (LOV) domains (e.g., EL222, YtvA, aureochrome-1, AsLOV2), blue light-using flavin adenine dinucleotide (FAD) (BLUF) domains (e.g., PixD, AppA, BLrP1, PAC, BlsA), cryptochrome domains (e.g., Cry2), fluorescent protein domains (e.g., Dendra, Dronpa, FusionRed, Kohinoor and Citrine) and phytochromes (e.g., PhyB, CPhl, BphP, Phyl, PixJ, Ac-NEO1). In some aspects, the light-responsive domain is a light oxygen voltage (LOV) domain, e.g., AsLOV2, the LOV2 domain from Avena sativa Phototropin 1.

A light responsive domain, such as “light oxygen voltage 2 domain” or “LOV2 domain”, can be naturally occurring or non-naturally occurring (e.g., engineered). For example, the LOV domain can be isolated (e.g., from a natural source), recombinant or synthetic. Examples of LOV domains that are suitable for use in the fusion proteins and methods described herein are known in the art and include variants of naturally occurring LOV domains (e.g., variants having at least about 70%, about 75%, about 80%, about 85%, about 90, about 95%, about 96%, about 97%, about 98% or about 99% identity to a naturally occurring LOV domain), such as AsLOV2, the LOV2 domain from Avena sativa Phototropin.

As used herein, the term “sequence identity” means that two nucleotide or amino acid sequences, when optimally aligned, such as by the programs GAP or BESTFIT using default gap weights, share at least 50% sequence identity, e.g., at least 70% sequence identity, or at least 75% sequence identity, or at least 80% sequence identity, or at least 85% sequence identity, or at least 90% sequence identity, or at least 95% sequence identity, or at least 98% sequence identity, or at least about 99% sequence identity or more. For sequence comparison, typically one sequence acts as a reference sequence (e.g., parent sequence) to which test sequences are compared. Unless otherwise indicated, the sequence identity comparison can be examined throughout the entire length of a sequence (e.g., reference sequence, test sequence), or within a desired fragment of a given sequence (e.g., reference sequence, test sequence). In some embodiments, sequence identity of a test sequence and an indicated reference sequence is determined over the entire length of the test sequence. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server).

Typically, default program parameters can be used to perform the sequence comparison, although customized parameters can also be used.

Because the light-responsive domain and, hence, the fusion protein, is light responsive, exposure to light induces a conformational change that alters an activity of the fusion protein. In some aspects, the conformational change of the fusion protein (typically, the light-responsive domain of the fusion protein) will be induced by visible light (e.g., from about 400-nm to 700-nm light). In particular aspects, the conformational change will be induced by blue light (e.g., from about 380-nm to about 500-nm light, in particular, about 450-nm light), red light (e.g., from about 620-nm to about 750-nm light) or far-red light (e.g., from about 710-nm to about 850-nm light). In other aspects, the conformational change will be induced by infrared light (e.g., from greater than 700-nm to about 1-mm light). LOV domains, BLUF domains, cryptochromes and fluorescent proteins, for example, are typically responsive to blue light, and phytochromes, for example, are typically responsive to red light and far-red light. The C-terminal Jα helix of AsLOV2, in particular, undocks and unfolds upon excitation with blue light (e.g., λmax=450 nm), resulting in a substantial increase in the distance between the N- and C-termini of AsLOV2, which are typically within less than 10 Å of one another in the absence of light.

Another embodiment is a method of altering an activity of a fusion protein comprising a light-responsive domain (e.g., a LOV domain, such as AsLOV2, the LOV2 domain from Avena sativa Phototropin 1) and a heterologous peptide component. The method comprises exposing the fusion protein to light that induces a conformational change in the fusion protein, thereby altering an activity of the fusion protein. The conformational change alters an activity of the fusion protein and, in some aspects, the activity is a binding activity selected from an in vitro binding activity and an in vivo extracellular binding activity.

The present invention is directed to protein constructs with the ability to induce and control reversible liquid-liquid phase separation, both globally and at specific subcellular locations. This system reveals that the location within the phase diagram can be used to dictate the material state of phase-separated IDR clusters, ranging from dynamic liquid droplets to arrested but reversible gels, which can over time mature into irreversible aggregates.

In the present invention, systems and methods are provided that utilize protein constructs with at least two regions fused to each other: (i) a light sensitive region containing a first segment (e.g., a protein sensitive to at least one wavelength of light) and (ii) a functional region containing a second segment (e.g., a low complexity sequence (LCS), an intrinsically disordered protein region (IDR), or one or more repeatable sequences).

Among the many different possibilities contemplated, a protein construct may also advantageously contain a desired protein to purify, or a fluorophore. In some embodiments, the second segment is an intrinsically disordered protein region (IDR). In some embodiments, the protein sensitive to at least one wavelength of light used in the first segment contains a protein that is sensitive to visible light. In some embodiments, the protein sensitive to at least one wavelength of light used in the first segment is Cry2, Cry2olig, PhyB, PIF, light-oxygen-voltage sensing (LOV) domains, or Dronpa. It is contemplated that these protein constructs will be configured such that after being introduced into a living cell, typically through transfection with DNA encoding for the protein construct, which is then translated into the protein by the native cellular machinery, exposing the living cell with the protein construct to certain wavelengths of light will induce the protein constructs within the living cell to cluster. It is further contemplated that if these protein constructs contain cleavage tags, such as self-cleaving tags, Human Rhinovirus 3C Protease (3C/PreScission), Enterokinase (EKT), Factor Xa (FXa), Tobacco Etch Virus Protease (TEV), or Thrombin (Thr), then after a first induction, it may be advantageous to cleave and induce clustering again.

The light sensitive region typically includes a first segment comprising at least one protein sensitive to at least one wavelength of light. In preferred embodiments, this segment includes Cry2, Cry2olig, PhyB, PIF, light-oxygen-voltage sensing (LOV) domains, or Dronpa. In other embodiments, the segment includes a protein sensitive to a visible wavelength of light, typically including wavelengths from about 400 nm to about 800 nm.

The functional region, which is fused to the light sensitive region, may include a second segment, the second segment comprising one or more low complexity sequences, one or more intrinsically disordered protein regions (IDRs), one or more synthetic or natural nucleic acid binding domains, or at least one repeatable sequence, the repeatable sequence comprising a linker fused to at least one additional gene encoding at least one protein sensitive to at least one wavelength of light. In preferred embodiments, the protein construct comprises an IDR, where the IDR is a portion of a first protein of, for example but not limited to, FUS, Ddx4, or hnRNPA1. Suitable IDRs also include but are not limited to shortened IDRs, for example shortened by over 50% to 93 amino acids. One example of a short IDR for use in the embodiments herein is “short FUS” also denoted as “sFUS”. Numerous IDRs are known in the art for use in the methods and synthetic proteins described herein. Useful disordered domains can be identified using the software tool IUPred (available from Eötvös Lorand University, known as “ELTE”) which predicts regions of disorder, or other methods known in the art for identifying disordered sequences.

An example of the protein construct was produced by fusing the “sticky” IDR from various proteins to the photolyase homology region (PHR) of Arabidopsis thaliana Cry2, a light-sensitive protein which is known to self-associate upon blue light exposure. This IDR-Cry2 fusion protein would recapitulate the modular domain architecture of many phase separating proteins, but confer tunable light-dependence to its multivalent interactions.

To facilitate a better understanding of the present invention, the following examples of certain aspects of some embodiments are given. In no way should the following examples be read to limit, or define, the entire scope of the invention.

Production of Light-Controlled Membraneless Organelles in Yeast.

To reversibly control metabolic flux in microbes, we first established a method for inducible synthetic organelle formation in which the yeast population exhibits reversible, visible, inducible clusters. We created optogenetic droplets that allow for inducible formation of synthetic organelles in response to light condition. Cry2/Cry2olig proteins oligomerize in response to 465 nm light. PixE proteins bind to PixD dimers to form a PixE₅PixD₁₀ complex in the dark and break apart under blue light stimulation. An intrinsically disordered domain, such as FUS, can be added to both systems to enable more robust, reversible clustering.

To minimize cell-to-cell variability it is preferable for the individual cells of a culture to demonstrate similar clustering in the illuminated and dark states. Light-controlled phase separation can exhibit substantial concentration dependence in both the kinetics and extent of clustering. At concentrations that are too low, clusters do not form regardless of light signal. At concentrations that are too high, clusters are constitutively formed and do not respond to light

To overcome this variability, we developed a simple and flexible strategy to ensure similar expression levels in each cell as well as tunable expression levels via antibiotic selection. We placed the clustering tags, which were also fused to fluorescent proteins for visualization, under a medium strength yeast promoter (P_{ADH1}) and integrated multiple copies of the construct into the yeast genome as previously described. We generalize the number of copies of this plasmid in a yeast strain by specifying a [Zeo]ₘₐₓ, corresponding to the maximum concentration of zeocin the yeast can grow on. A [Zeo]ₘₐₓ of 400 mg/L corresponds to 1-2 copies; a [Zeo]ₘₐₓ of 800 mg/L corresponds to 3-4 copies; a [Zeo]ₘₐₓ of 1,200 mg/L corresponds to 5-6 copies.

Assembly of DNA Constructs.

Ligations and one-step isothermal assembly reactions were performed using methods known in the art.

Expression of optoDroplets in Saccharomyces cerevisiae.

A 2μ plasmid (YEZ53) containing a fusion of FUS^{N}, the FusionRed fluorescent protein and Cry2 was constructed for expression in Saccharomyces cerevisiae. Light-dependent changes in oligomerization of this fusion protein were observed in Saccharomyces cerevisiae cells demonstrating that the optoDroplet system is functional in yeast. Some cell-to-cell variability in droplet formation was observed due to variability in gene expression and protein levels.

Selection Strategy for Obtaining Homogeneous Clustering.

To overcome cell-to-cell variability in droplet formation and identify ideal protein levels for light-induced organelle assembly and homogeneous responses in most cells in a colony, a genome integration and selection strategy was employed using the antibiotic zeocin. Zeocin-resistant optogenetic cassettes were integrated into yeast cells and transformed cells were replica plated on plates containing different zeocin concentrations to obtain optoDroplet expression levels that supported light-switchable droplet formation in most or all cells within a colony.

Cassettes were constructed for expression of either the optoDroplet or optoCluster systems driven by the medium-strength P_{ADH1} promoter as well as a zeocin resistance marker (plasmid pNS1 and pNS3, Table 1), and integrated variable numbers of copies of the construct into δ-sites of the yeast genome. To further characterize the dynamics and reversibility of photoswitchable organelle formation, optoDroplet and optoCluster colonies were selected with a [Zeo]max of 800 mg 1-1 and imaged FusionRed localization by confocal microscopy in response to sequences of darkness and blue illumination. Strains expressing FusionRed-Cry2 or FusionRed-Cry2olig without the FUS^{N} IDR tag did not exhibit robust clustering at any [Zeo]max level.

Light-Induced Clustering Shifted Flux Toward a Desired Product on Illumination.

The optoDroplets exhibited the cleanest change from diffuse to clustered localization on light stimulation. In contrast, optoClusters exhibited some clusters in un-illuminated cells but also exhibited more overall redistribution into clusters on illumination. Cry2olig shows an increased propensity to cluster compared to Cry2. Similar results were obtained with the inverse PixELL system. On the basis of the observation that PixELL clusters contain PixD and PixE in a 2:1 stoichiometry, we first integrated a single copy of FUS^{N}-Citrine-PixE driven by the PPGK1 promoter into the HIS3 locus, and then integrated a variable number of copies of PADH1 driven FUSN-FusionRed-PixD into δ-sites (YEZ232, Tables 1 and 2). Colonies having [Zeo]max of 1,200 mg 1-1 exhibit robust PixD/PixE clustering that dissociate after blue light stimulation. As expected, both PixD and PixE constructs were required for clustering, as strains expressing only one or the other showed only diffuse localization.

We further validated that optoDroplets, optoClusters and PixELLs are each functional in two yeast strains commonly used in cell biology and metabolic engineering studies, BY4741 and CEN.PK2-1C. For each optogenetic system, we quantified the number of clusters formed on illumination and their assembly/disassembly kinetics after illumination changes. Taken together, our results show that the assembly and disassembly of membraneless organelles is robustly triggered with light across a colony of engineered budding yeast cells.

Light-triggered deoxyviolacein flux using optoClusters. The ability to induce the formation of synthetic membraneless organelles could have enormous potential for metabolic engineering, enabling the on-demand compartmentalization of metabolic enzymes and thus control of metabolic flux. To demonstrate this use for metabolic engineering in a controlled model system, we set out to control the flux through the deoxyviolacein pathway.

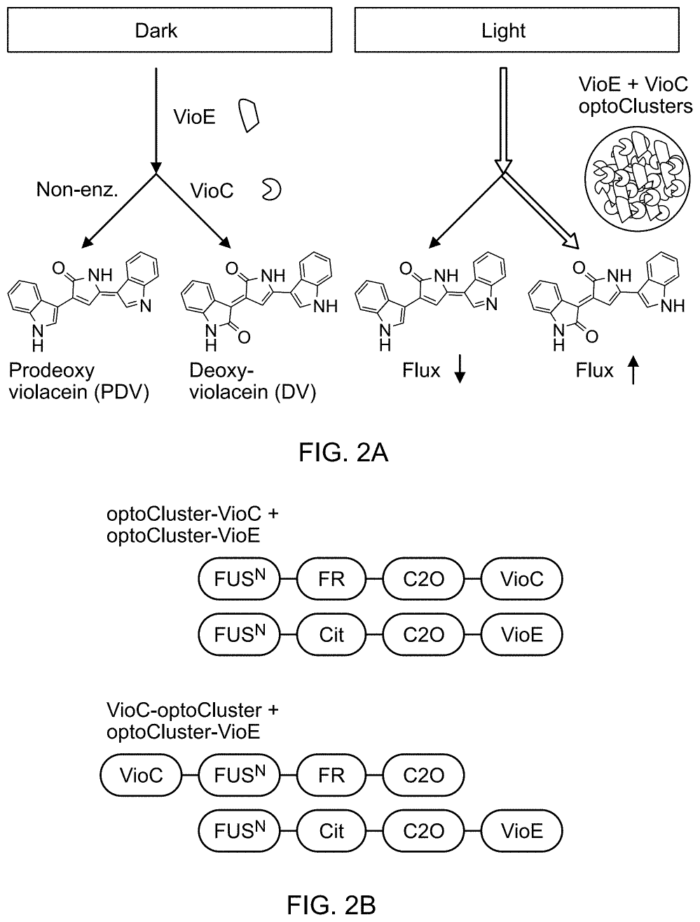

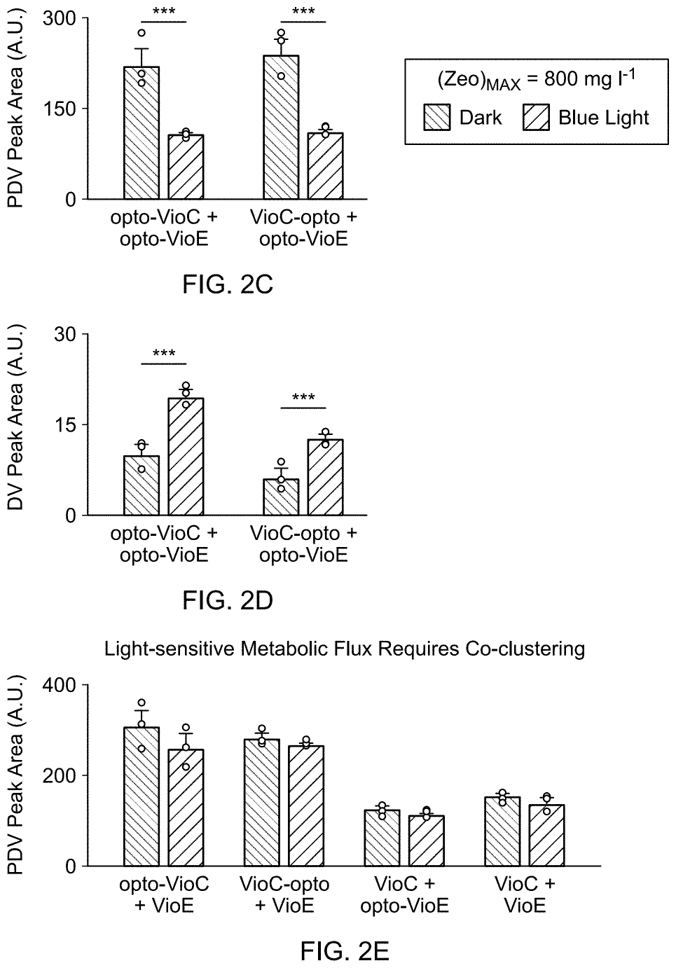

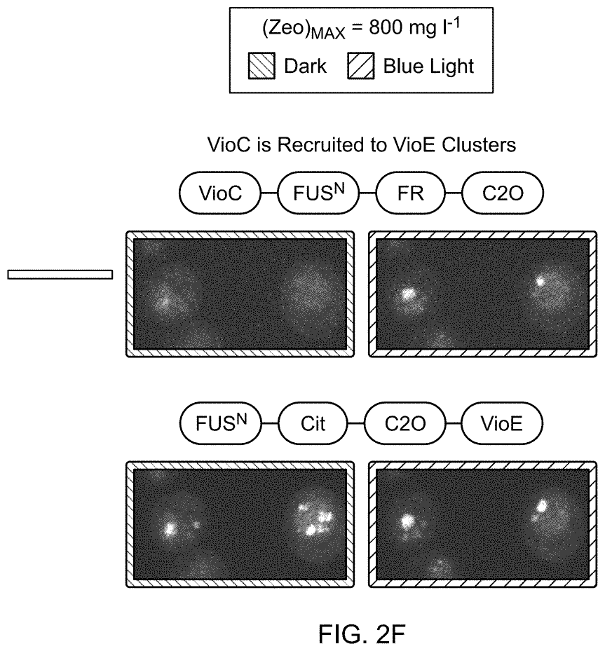

The deoxyviolacein pathway produces two distinct end products depending on the level of activity of two enzymes: VioE and VioC (FIG. 2A). VioE catalyzes the formation of an intermediate, protodeoxyviolaceinate (PTDV), which is then converted by VioC to the pink-colored product deoxyviolacein. Alternatively, PTDV can be spontaneously oxidized to a green product, prodeoxyviolacein (PDV). Both products, PDV and deoxyviolacein, can be detected by chromatographic methods. This ease of product quantification makes the deoxyviolacein pathway an ideal platform for assessing metabolic flux control by light inducible enzyme clustering.

Inducing the co-localization of VioE and VioC, shifts flux from PDV to deoxyviolacein production. VioE and VioC were fused to the components of our optoCluster system (FIG. 2A). Yeast strain (YNS21) constitutively expresses VioA and VioB (Table 2). We then integrated several copies of a cassette containing VioE-optoCluster and VioC-optoCluster fusions, driven by PADH1, into δ-sites of YNS21 (Tables 1 and 2). Both N and C terminal orientations were tested for the optoCluster/enzyme fusions, leading to a total of four yeast strains (YNS34, YNS34-cterm, YNS36, YNS36-cterm) (Table 2). We then screened several colonies of each transformation with various [Zeo]max levels for light-dependent changes in PDV production. The two strains expressing VioE-optoCluster (YNS34-cterm, YNS36-cterm) failed to produce any detectable deoxyviolacein in either light or dark conditions, suggesting that VioE is nonfunctional with the optoCluster domains fused to its C terminus. However, strains co-expressing optoCluster-VioE and either optoCluster-VioC (YNS34) or VioC-optoCluster (YNS36) exhibit approximately a two-fold increase in deoxyviolacein production and a corresponding decrease in PDV production when incubated under continuous blue light, relative to their production levels in the dark (FIGS. 2B-D). Inside a cluster, the PTDV intermediate produced by VioE has an increased likelihood of encountering co-clustered VioC, leading to enhanced deoxyviolacein production. This enhanced conversion reduces steady-state PTDV levels, decreasing the production of the alternative PDV product. A series of control experiments was conducted to confirm that flux redirection was due to co-clustering of both enzymes rather than a clustering-induced change in the function of VioC or VioE alone. No light-dependent change in product formation was observed in strains expressing VioC-optoCluster and un-clustered VioE, unclustered VioC and optoCluster-VioE, or VioE and VioC without clustering tags. The total protein levels of VioC and VioE were not changed by light or dark incubation in either of two VioC/VioE-optoCluster strains (YNS34 and YNS36).

Live-cell microscopy was used to verify that VioE-VioC clusters were light-switchable (FIG. 2F). VioE formed constitutive clusters even without light exposure, probably due to synergy between VioE's innate tendency to oligomerize and the FUS^{N} tag. In contrast, VioC's clustering was light-inducible: VioC was diffuse in the dark, shifting to clusters that co-localized with VioE on light stimulation (FIG. 2F, right panels). Taken together, our results show a shift in metabolic flux from PDV to deoxyviolacein production driven by enhanced substrate conversion within light-induced VioE-VioC clusters.

Colony screening, light stimulation and deoxyviolacein/PDV product analysis were repeated in analogous strains using the optoDroplet system (YNS34drop, YNS36drop) and with Cry2olig-VioC/VioE that lacked the FUS^{N} tag (YEZ250), but did not observe an increase in deoxyviolacein production under blue light. As the FUS^{N} tag and Cry2olig variant both serve to increase the extent of light-induced clustering, these data demonstrate that the strongest-clustering optogenetic variants are best-suited for shifting metabolic flux.

Light-Suppressed Deoxyviolacein Flux Using PixELLs.

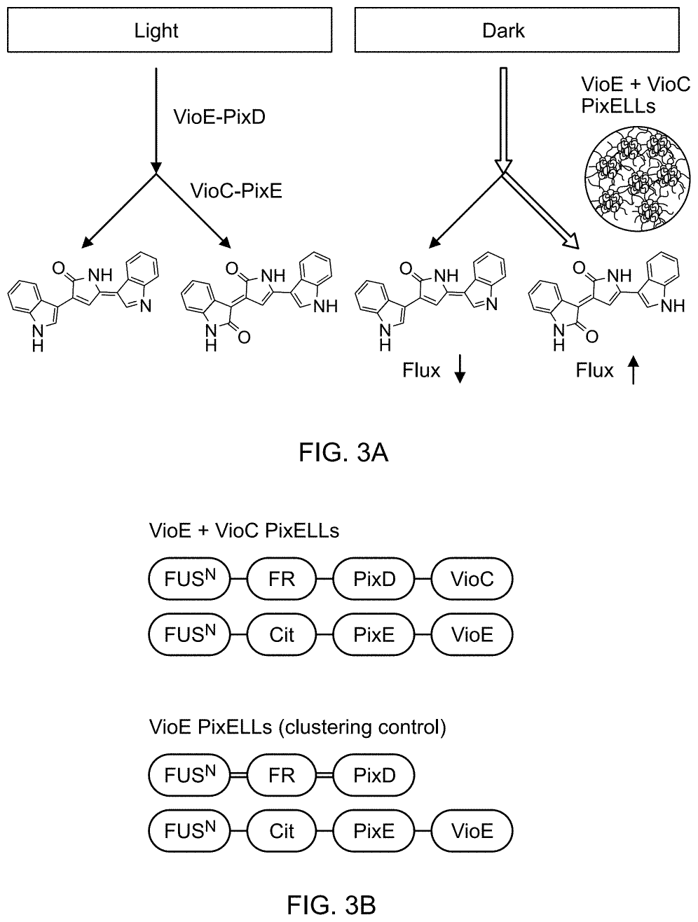

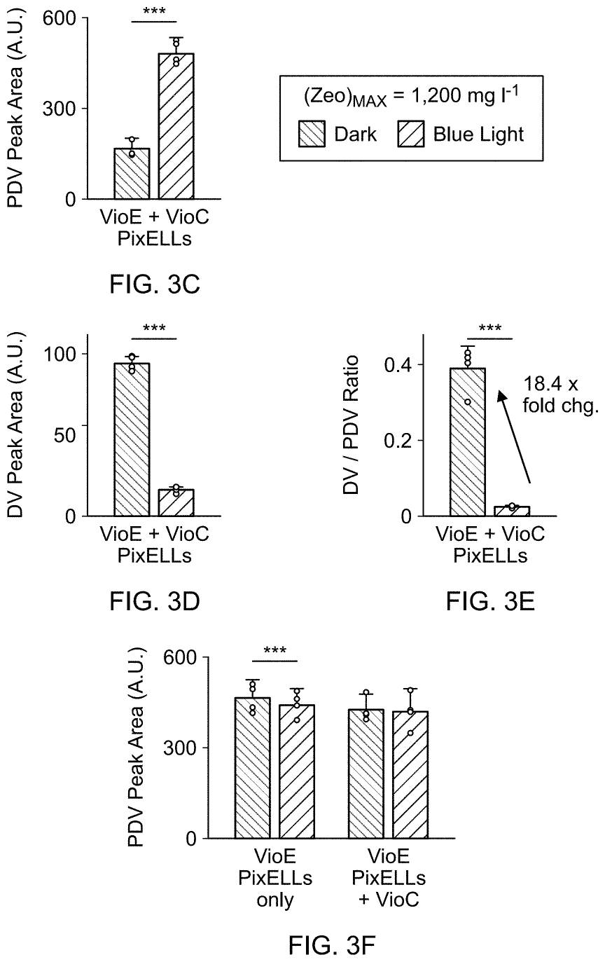

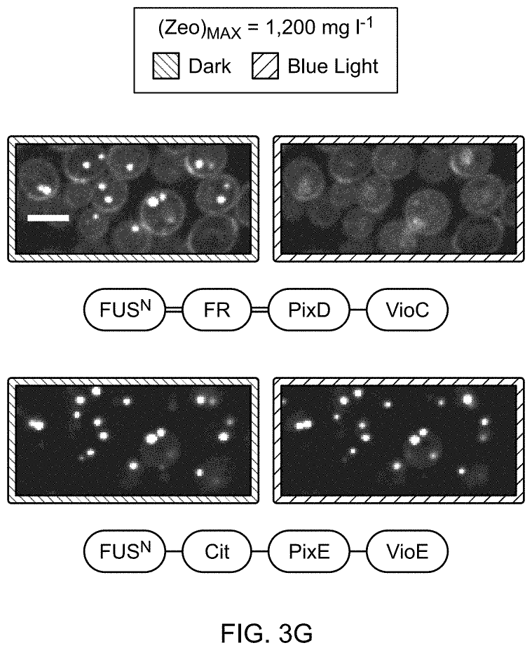

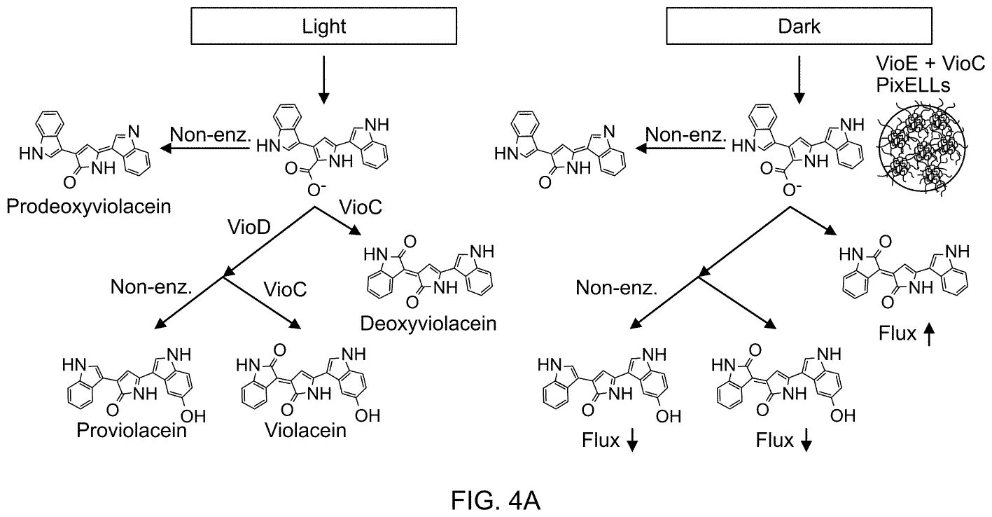

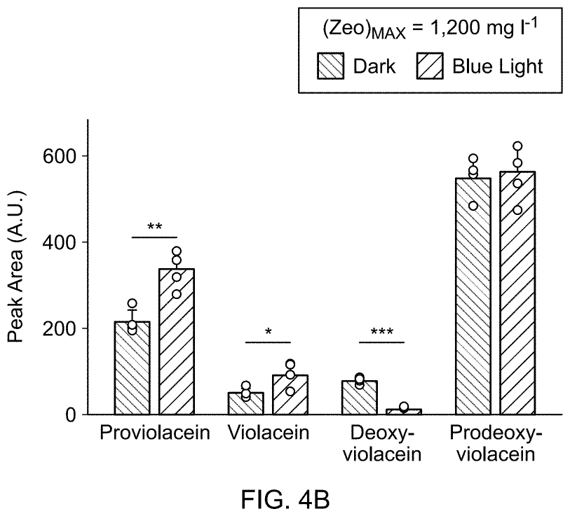

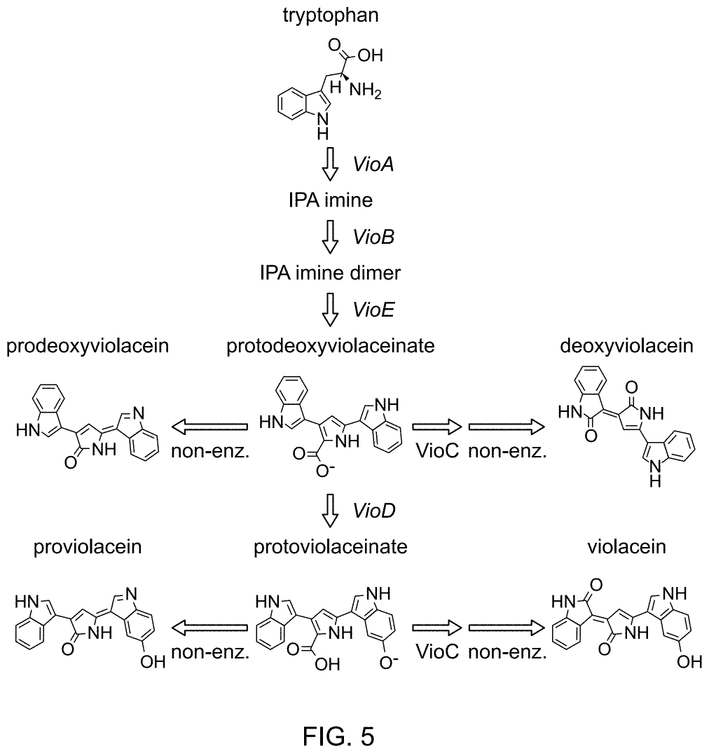

Light dissociable enzyme clusters have the benefit of enhancing flux toward a desired product on a shift from light to darkness, which may be easier to achieve in high-cell-density fermentations and in large-scale bioreactors. Furthermore, having both light assembled and light-dissociated organelles in the same strain could enable bidirectional control, shifting cells from growth promoting metabolism to an engineered pathway by changing light conditions. The PixELL system was used to generate light-dissociable metabolic organelles. (FIG. 3A). Starting from YEZ282 (with VioA/VioB in the LEU2 locus), we integrated one copy of FUS^{N}-Citrine-PixEVioE driven by the strong constitutive PPGK1 promoter into the HIS3 locus, and then integrated multiple copies of FUS^{N}-FusionRed-PixD-VioC into δ-sites to create strain YEZ257 (FIG. 3B). We found that YEZ257 colonies with a [Zeo]max of 1,200 mg 1-1 exhibited a pronounced metabolic shift between light and dark conditions (FIGS. 3C and 3D), exceeding the fold change observed previously with the optoCluster system. The best colony showed a 6.1 Å} 0.9-fold change in deoxyviolacein production and a corresponding decrease in PDV titer (FIGS. 3C and 3D), leading to an 18.4 Å} 4.5-fold change in deoxyviolacein-to-PDV ratio from light to dark conditions (FIG. 3E). This effect was not observed for colonies where [Zeo]max was 400, 800 or 1,600 mg 1-1, supporting the observation that the photoswitchable response is optimal at intermediate fusion protein expression levels, where light can efficiently assemble/disassemble clusters throughout the cell population.

As in the case of deoxyviolacein-producing optoClusters, it was confirmed that these light-dependent changes in metabolic flux reflect the assembly/disassembly of clusters containing VioC and VioE. Strains expressing VioE-only PixELLs in the presence or absence of un-clustered VioC (YEZ512 and YEZ281, respectively) did not exhibit light-dependent changes in PDV production (FIG. 3F). The metabolic shift was also not due to light-induced changes in protein expression, as there was no difference in VioC or VioE expression levels as a function of light stimulus (strains YEZ257 and YEZ281). Live-cell microscopy confirmed that VioE/VioC PixELLs were assembled in the dark and could be dissociated in blue light (FIG. 3G). Time-lapse imaging of strain YEZ257 revealed that blue light stimulation caused VioC to switch from a clustered to a diffuse subcellular distribution (FIG. 3G, left). In contrast, VioE remains clustered in both light and dark conditions (FIG. 3G, right). Together, these data demonstrate that PixELL-enzyme fusions are a powerful platform for darkness-triggered metabolic flux, complementing the light triggered flux of optoCluster-enzyme fusions. Some of the optogenetic tags described herein incorporate a 200 amino acid disordered domain (FUS^{N}), a fluorescent protein and a light-sensitive domain (for example, the FUS^{N}-FusionRed-PixD tag is 483 amino acids). Shortened variants were tested to determine if they might still retain strong light-dependent clustering and metabolic flux enhancement. We found that the FUS^{N} DR could be shortened by over 50% to 93 amino acids (termed ‘short FUS’ or sFUS) while retaining potent light-regulated PixELL clustering (strain YEZ555). Removing the fluorescent protein generated a final sFUS-PixD tag that is approximately half the size of our original tag (247 versus 483 amino acids). The resulting PixELL-expressing strain (YEZ553) was still able to generate a strong light-induced flux shift and further increased the maximum overall deoxyviolacein yield by 3.2-fold.

In some embodiments, the length of the fusion protein, IDR sequence, or light-switchable clustering domains may be modified to enhance metabolic flux. It may also be advantageous to more precisely control the subcellular localization of our optogenetic tools, which are expressed throughout the nucleus and cytosol and can cluster in either compartment. Adding subcellular localization tags (for example, nuclear export sequences or mitochondrial localization tags) can be used to increase yields by limiting clustering to subcellular compartments where the concentration of upstream metabolites is highest.

Light-Controlled Flux at an Enzymatic Branch Point.

Finally, the enzyme-catalyzed deoxyviolacein production competes with a nonenzymatic side pathway. However, many metabolic pathways have branch points where two enzymes compete for access to a single intermediate raising the question whether clustering would be effective at such a two-enzyme branch point. A branch point can be created by adding a single additional enzyme, VioD. VioD competes with VioC for the substrate PTDV, driving the formation of two other pigments: proviolacein and violacein. VioD driven by the PPGK1 promoter from a 2μ plasmid was inserted into strain YEZ257. It was found that flux through both enzymatic branches could be switched with light: proviolacein/violacein levels were highest in the light when VioE/VioC PixELLs were dissociated, and deoxyviolacein levels were highest in the dark.

However, unlike the results obtained from the linear pathway a change in PDV levels was not observed in the branched-enzyme scenario. PDV is produced nonenzymatically from PTDV, so the observation of constant PDV levels sought to extend the use of light-controlled metabolic organelles to a more complex scenario. In the deoxyviolacein pathway used so far, suggests that the PTDV intermediate levels are no longer changed by light-triggered clustering. This observation may reflect the balance of two competing effects. VioE-VioC clustering is expected to simultaneously increase the consumption of PTDV by VioC but decrease its encounter frequency with VioD; these two effects may balance such that combined flux through both enzymatic pathways is unchanged. Taken together, our data demonstrate that the light-induced assembly/disassembly of enzyme-containing membraneless organelles can be used to shunt metabolic flux toward a product of interest and away from competing branches. Similar deoxyviolacein results were observed with both light-induced optoClusters and darkness induced PixELLs, demonstrating that our results are robust to off-target, light-dependent processes such as photo-degradation of metabolites or unintended manipulation of endogenous light-sensitive biochemical reactions. In future studies, the bidirectional control afforded by these two systems could also be useful to enhance different sets of reactions under light and dark conditions, thereby reversibly switching cells between ‘growth’ and ‘production’ phases.

Reversible clustering activity can be obtained using intermediate range of fusion protein expression levels, for example, using a medium-strength promoter.

Construction of optoDroplet, optoCluster and PixELL Expressing Strains.

OptoDroplet and optoCluster yeast strains were created by integrating multiple copies of constructs containing different combinations of FUS^{N}, Cry2 and Cry2olig (pNS1, pNS2, pNS3, pNS4) fused to fluorescent proteins and selected on increasing levels of zeocin (400 mg/L, 800 mg/L, 1,200 mg/L, and 1,600 mg/L, which corresponded to an increasing number of integration events). The resulting strains were yNS47, yNS48, yNS49, and yNS50, respectively. Only constructs with the FUS^{N} tag (pNS1, pNS3) formed visible phase-separated bodies when induced with light. To construct PixELL-expressing strains, we integrated a single copy of FUS^{N}-Citrine-PixE (EZ-L498 to make YEZ231) into the HIS3 locus and multiple copies of FUS^{N}-FusionRed-PixD (EZ-L499 to make YEZ232, selected on 1,200 mg/L zeocin) into the δ-sites in the yeast genome. No phase separation was observed when only one component, PixD or PixE alone, was used (YEZ234).

Screening of Other Clustering Constructs.

To test other light-inducible clustering tags, plasmids pNP1-Drop, pNP3-Drop, pNP7, and EZ-L477 were constructed representing all combinations of fusions of FUS^{N}-FusionRed-Cry2 and FusionRed-Cry2olig with either VioE or VioC. Screening of 24 colonies of yNS21+pNP1-Drop+pNP3-Drop (yNS34drop), 24 colonies of yNS21+pNP2-Drop+pNP3-Drop (yNS36drop), and 24 colonies of yNS21+pNP7+EZ was conducted. L477 (YEZ250). None of these combinations yielded a higher production of DV in the light than in the dark.

OptoClusters and PixELLs exhibit stronger metabolic shifts than optoDroplets. The optoCluster system includes an additional point mutation that favors Cry2 oligomerization and clustering. Also, the PixELL system is made up of two Pix proteins, so whichever of the two is limiting in expression tends to exhibit near-complete redistribution in/out of clusters. These differences could lead optoDroplets to have a lower total shift from a diffuse to clustered enzyme distribution. OptoDroplet enzyme expression can be optimized by screening additional colonies or testing additional [Zeo]ₘₐₓ values to shift metabolic flux.

Deoxyviolacein Pathway Control Using PixELLs.

To redirect flux towards DV with the PixELL system, we integrated EZ-L528 (for expression of VioA and VioB, required to produce the IPA imine dimer precursor metabolite from tryptophan) into BY4741 to make YEZ282 (Tables 1 and 2). We then integrated one copy of a FUS^{N}-Citrine-PixE-VioE fusion (EZ-L526) under a strong, constitutive promoter, P_{PGK1}, to make YEZ255. We expressed various levels (at 400 mg/L, 800 mg/L, 1,200 mg/L, and 1,600 mg/L of zeocin) of a FUS^{N}-FusionRed-PixD-VioC fusion (EZ-L527) by using δ-integration (YEZ257, see Tables 1 and 2). For colonies where [Zeo]ₘₐₓ=1,200 mg/L, we observed a higher level of DV production when the culture was grown in the dark than when the DV production when the same culture was grown in the light (FIG. 3D). The best colony showed a 6.1-fold change from light to dark conditions with consistent decreases in PDV titer (FIG. 3D). This effect was not observed for colonies where [Zeo]ₘₐₓ was 400 mg/L, 800 mg/L or 1,600 mg/L of zeocin. We controlled for the effects of clustering by integrating EZ-L499 into YEZ255, resulting in YEZ281, a strain that clusters PixD and PixE upon blue light stimulation but lacks VioC and thus produces no DV. We also added pNS7 to YEZ281 to control for constitutive non-localizing VioC control, making YEZ512.

Diverting Flux Away from VioD Using PixELLs.

To test the effect of the PixELL system on a metabolic branchpoint containing a competing enzyme, we added VioD to the existing system. We added EZ-L859 to YEZ257 to make YEZ511. We saw that in YEZ511, the entire system produced more products of the violacein pathway (PDV, DV, proviolacein, and violacein). However, we also saw the intended effect, which was a shift from more DV production in the light to more proviolacein and violacein production in the dark. This dependence on light condition of proviolacein and violacein production was not seen in control strains.

Shortening the FUS^{N} Tag for Diverting Flux Using PixELLs.

As the size of the tags are large and could complicate protein activity and/or expression, we tested a tag of reduced size for chemical production. We did this by first limiting the size of the FUS^{N} domain to the first 93 amino acids. We named this iteration sFUS. We first tested to see how sFUS functions with fluorescent proteins. We added EZ-L767 (sFUS-FR-PixD) to YEZ231 and selected on 1,200 mg/L of zeocin to make YEZ555. We then wanted to test minimizing the tag on DV production. We did this by both shortening FUS to sFUS and removing the fusion red protein from the VioC expression construct. We integrated EZ-L786 into YEZ255 to make YEZ553 and selected on 1,200 mg/L of zeocin to make YEZ553. We controlled for the effects of clustering by integrating EZ-L767 into YEZ255, resulting in YEZ554, a strain that clusters PixD and PixE upon blue light stimulation but lacks VioC and thus produces no DV.

Assessing and Correcting for Violacein Product Photobleaching.

An optogenetic system requires continuous illumination with blue light which raises the possibility of light-induced photobleaching or degradation. To measure the photobleaching and/or degradation of PDV, DV, proviolacein, and violacein under blue light stimulation, we measured the production of PDV, DV, proviolacein, and violacein in strains constitutively expressing violacein enzymes without optogenetic control (yNS51, MZW342, MZW375, MZW377, and MZW378) under lit and dark conditions. In four of these strains (MZW342, MZW375, MZW377 and MZW378), expression of the violacein pathway enzymes was under the control of a β-estradiol inducibler promoter. We thus added β-estradiol to a final concentration of 1μM throughout the fermentation. For all light stimulation experiments we used the same blue light source under identical conditions.

We found that DV is degraded slowly and at a constant rate by blue light, so that illuminated samples always exhibited a proportionally smaller DV peak by HPLC.

Individual points represent yNS51, MZW342, MZW375, MZW377, and MZW378, five strains with different DV production levels. We thus normalized all DV measurements performed after blue light illumination using the standard curve produced by these control strains. We observed no photobleaching by blue light for PDV, proviolacein, or violacein in these assays. No differences in growth rate or maximum optical density were observed from these strains when cultured in the light or dark.

Analyses of Cluster Number & Assembly/Disassembly Kinetics.

We observed light-dependent organelle formation in yeast made to express three of our optogenetic systems: optoClusters, optoDroplets and PixELLs. Yet in each of these cases, the extent and timescale of clustering differed, an observation that we sought to describe more quantitatively using live-cell imaging in each case. We imaged yeast strains yNS47 (OptoDroplets), yNS49 (OptoClusters) and YEZ232 (PixELLs) in the FusionRed channel during cycles of 450 nm blue light illumination or darkness. We then quantified the extent of clustering by analyzing the number of clusters per cell and the kinetics of cluster assembly/disassembly using changes in the pixel-to-pixel signal-to-noise ratio (SNR, which measures the homogeneity of cluster intensities (i.e., lower SNR=more clustering). We found that on average we observed between 1-4 clusters per cell across these three systems, with fewer PixELLs and OptoDroplets per cell, and more OptoClusters per cell under clustering conditions. However, the number of clusters depends on a large number of parameters, including the length of time of clustering (due to processes such as ripening and fusion events) and the expression level of the constructs, so these results should be taken as indicative of results in our conditions, not universal properties of these optogenetic tools.

We also measured the kinetics of cluster assembly/disassembly, observing fast light-induced changes in all three systems. These changes worked in opposing directions depending on the optogenetic system used. For instance, we observed light-induced assembly over ˜5 min in OptoCluster/OptoDroplet cells, and light-induced disassembly within 30 sec in PixELL-expressing cells. In contrast, dark-induced reversion occurs on different timescales for each optogenetic system: PixD switches back to its dark-state conformation with a half-life of ˜5 sec², whereas Cry2 switches back in ˜2 min and Cry2olig in˜20 min³. Matching these optogenetic dark-state kinetics, we found that Cry2-based OptoDroplets dissociated in minutes, Cry2olig-based OptoClusters were not fully dissociated even after 30 min in the dark, and PixD-based PixELLs reassembled in minutes after dark incubation.

TABLE 1
Plasmids

| Plasmid | Position 1* | Position 2* | Position 3* | Marker | Vector type |
|---|---|---|---|---|---|
| pJLA121⁰³⁰¹ | P_{PGK1}_Multiple Cloning Sequence (MCS)_T_{CYC1} | EMPTY | EMPTY | URA3 | 2μ |
| pYZ12-B | EMPTY | EMPTY | EMPTY | HIS3 | Integration |
| | | | | | into HIS3 |
| | | | | | Locus |
| pYZ23 | EMPTY | EMPTY | EMPTY | Zeocin | Integration |
| | | | | | into δ-sites |
| pNS1 | P_{ADH1}_FUS^{N}_FusionRed_Cry2WT_T_{ACT1} | EMPTY | EMPTY | Zeocin | Integration |
| | | | | | into δ-sites |
| pNS2 | P_{ADH1}_FusionRed_Cry2WT_T_{ACT1} | EMPTY | EMPTY | Zeocin | Integration |
| | | | | | into δ-sites |
| pNS3 | P_{ADH1}_FUS^{N}_FusionRed_Cry2Olig_T_{ACT1} | EMPTY | EMPTY | Zeocin | Integration |
| | | | | | into δ-sites |
| pNS4 | P_{ADH1}_FusionRed_Cry2Olig_T_{ACT1} | EMPTY | EMPTY | Zeocin | Integration |
| | | | | | into δ-sites |
| pNS5 | P_{TEF1}_VioB_T_{ACT1} | P_{PGK1}_ViOA_T_{CYC1} | EMPTY | HIS3 | Integration |
| | | | | | into HIS3 |
| | | | | | Locus |
| pNS6 | P_{TEF1}_VioB_T_{ACT1} | P_{PGK1}_ViOA_T_{CYC1} | P_{GPD}-_VioE_T_{ADH1} | HIS3 | Integration |
| | | | | | into HIS3 |
| | | | | | Locus |
| pNS7 | P_{TEF1}_VioC_T_{ACT1} | EMPTY | EMPTY | URA3 | 2μ |
| pNS8 | P_{TEF1}_VioE_T_{ACT1} | EMPTY | EMPTY | URA3 | CEN6_ARS4 |
| pNS9 | P_{TEF1}_VioC_T_{ACT1} | | | | CEN6_ARS4 |
| pNP1 | P_{ADH1}_FUS^{N}_Citrine_Cry2Olig_VioC_T_{ACT1} | EMPTY | EMPTY | Zeocin | Integration |
| | | | | | into δ-sites |
| pNP2 | P_{ADH1}_VioC_FUS^{N}_Citrine_Cry2Olig_T_{ACT1} | EMPTY | EMPTY | Zeocin | Integration |
| | | | | | into δ-sites |
| pNP3 | P_{ADH1}_FUS^{N}_FusionRed_Cry2Olig_VioE_T_{ACT1} | EMPTY | EMPTY | Zeocin | Integration |
| | | | | | into δ-sites |
| pNP4 | P_{ADH1}_VioE_FUS^{N}_FusionRed_Cry2Olig_T_{ACT1} | EMPTY | EMPTY | Zeocin | Integration |
| | | | | | into δ-sites |
| pNP1-Drop | P_{ADH1}_FUS^{N}_Citrine_Cry2_VioC_T_{ACT1} | EMPTY | EMPTY | Zeocin | Integration |
| | | | | | into δ-sites |
| pNP2-Drop | P_{ADH1}_VioC_FUS^{N}_Citrine_Cry2_T_{ACT1} | EMPTY | EMPTY | Zeocin | Integration |
| | | | | | into δ-sites |
| pNP3-Drop | P_{ADH1}_FUS^{N}_FusionRed_Cry2_VioE_T_{ACT1} | EMPTY | EMPTY | Zeocin | Integration |
| | | | | | into δ-sites |
| pNP7 | P_{ADH1}_FR_Cry2Olig_VioE_T_{ACT1} | EMPTY | EMPTY | Zeocin | Integration |
| | | | | | into δ-sites |
| EZ-L176 | P_{GPD1}_FUS^{N}_FusionRed_Cry2_T_{ACT1} | EMPTY | EMPTY | URA3 | 2μ |
| EZ-L477 | P_{ADH1}_Citrine_Cry2Olig_VioC_T_{ACT1} | EMPTY | EMPTY | Zeocin | Integration |
| | | | | | into δ-sites |
| EZ-L498 | P_{PGK1}_FUS^{N}_Citrine_PixE_T_{CYC1} | EMPTY | EMPTY | HIS3 | Integration |
| | | | | | into HIS3 |
| | | | | | Locus |
| EZ-L499 | P_{ADH1}_FUS^{N}_FusionRed_PixD_T_{ACT1} | EMPTY | EMPTY | Zeocin | Integration |
| | | | | | into δ-sites |
| EZ-L526 | P_{PGK1}_FUS^{N}_Citrine_PixE_VioE_T_{CYC1} | EMPTY | EMPTY | HIS3 | Integration |
| | | | | | into HIS3 |
| | | | | | Locus |
| EZ-L527 | P_{ADH1}_FUS^{N}_FusionRed_PixD_VioC_T_{ACT1} | EMPTY | EMPTY | Zeocin | Integration |
| | | | | | into δ-sites |
| EZ-L528 | P_{TEF1}_VioB_T_{ACT1} | P_{PGK1}_VioA_T_{CYC1} | EMPTY | LEU2 | Integration |
| | | | | | into LEU2 |
| | | | | | Locus |
| EZ-L767 | P_{ADH1}_sFUS_FusionRed_PixD_T_{ACT1} | EMPTY | EMPTY | Zeocin | Integration |
| | | | | | into δ-sites |
| EZ-L786 | P_{ADH1}_sFUS_PixD_VioC_T_{ACT1} | EMPTY | EMPTY | Zeocin | Integration |
| | | | | | into δ-sites |
| EZ-L859 | P_{PGK1}_VioD_T_{CYC1} | EMPTY | EMPTY | URA3 | 2μ |

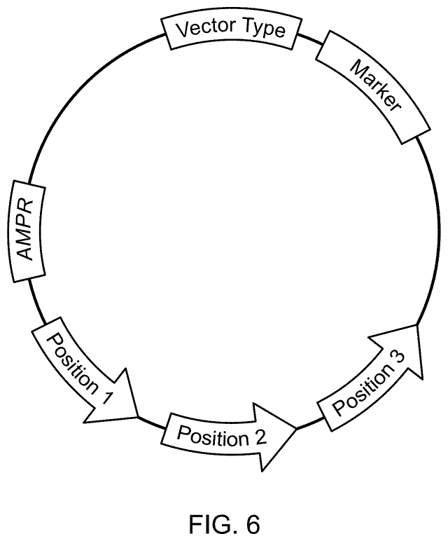

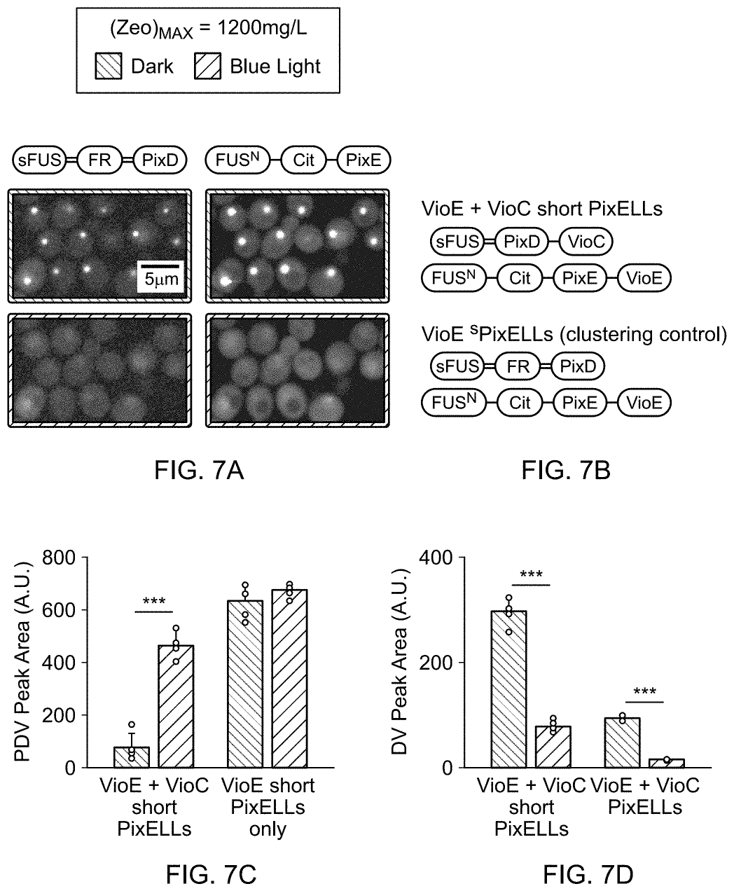

All vectors are constructed according to the map shown in FIG. 6.

TABLE 2
Yeast Strains

| Strain Name | Genotype | Source |
|---|---|---|
| BY4741 | S288C MATa his3Δ1 leu2Δ0 met15Δ0 ura3Δ0 | Brachmann et al. |
| CEN.PK2-1C | MATa his3Δ1 leu2-3_112 trp1-289 ura3-53 | Entian et al. |
| YNS21 | BY4741 HIS3::P_{TEF1}_VioB_T_{ACT1} + P_{PGK1}_VioA_T_{CYC1} | Described herein |
| YNS34 | YNS21 | Described herein |
| | YARCdelta5::P_{ADH1}_FUS^{N}_Citrine_Cry2Olig_VioC_T_{ACT1}; | |
| | YARCdelta5:: | |
| | P_{ADH1}_FUS^{N}_FusionRed_Cry2Olig_VioE_T_{ACT1} | |
| YNS34cterm | YNS21 | Described herein |
| | YARCdelta5::P_{ADH1}_FUS^{N}_Citrine_Cry2Olig_VioC_T_{ACT1}; | |
| | YARCdelta5:: | |
| | P_{ADH1}_VioE_FUS^{N}_FusionRed_Cry2Olig_T_{ACT1} | |
| YNS34Drop | YNS21 | Described herein |
| | YARCdelta5::P_{ADH1}_FUS^{N}_Citrine_Cry2_VioC_T_{ACT1}; | |
| | YARCdelta5::P_{ADH1}_FUS^{N}_FusionRed_Cry2_VioE_T_{ACT1} | |
| YNS36 | YNS21 | Described herein |
| | YARCdelta5::P_{ADH1}_VioC_FUS^{N}_Citrine_Cry2Olig_T_{ACT1}; | |
| | YARCdelta5:: | |
| | P_{ADH1}_FUS^{N}_FusionRed_Cry2Olig_VioE_T_{ACT1} | |
| YNS36cterm | YNS21 | Described herein |
| | YARCdelta5::P_{ADH1}_VioC_FUS^{N}_Citrine_Cry2Olig_T_{ACT1}; | |
| | YARCdelta5:: | |
| | P_{ADH1}_VioE_FUS^{N}_FusionRed_Cry2Olig_T_{ACT1} | |
| YNS36Drop | YNS21 | Described herein |
| | YARCdelta5::P_{ADH1}_VioC_FUS^{N}_Citrine_Cry2_T_{ACT1}; | |
| | YARCdelta5::P_{ADH1}_FUS^{N}_FusionRed_Cry2_VioE_T_{ACT1} | |
| yNS46 | BY4741 HIS3:: | Described herein |
| | (P_{TEF1}_VioB_T_{ACT1} + P_{PGK1}_VioA_T_{CYC1} + P_{GPD}_VioE_T_{ADH1}) | |
| yNS47 | CEN.PK2-1C | Described herein |
| | YARCdelta5::P_{ADH1}_FUS^{N}_FusionRed_Cry2WT_T_{ACT1} | |
| yNS47BY | BY4741 | Described herein |
| | YARCdelta5::P_{ADH1}_FUS^{N}_FusionRed_Cry2WT_T_{ACT1} | |
| yNS48 | CEN.PK2-1C | Described herein |
| | YARCdelta5::P_{ADH1}_FusionRed_Cry2WT_T_{ACT1} | |
| yNS48BY | BY4741 YARCdelta5::P_{ADH1}_FusionRed_Cry2WT_T_{ACT1} | Described herein |
| yNS49 | CEN.PK2-1C | Described herein |
| | YARCdelta5::P_{ADH1}_FUS^{N}_FusionRed_Cry2Olig_T_{ACT1} | |
| yNS49BY | BY4741 | Described herein |
| | YARCdelta5::P_{ADH1}_FUS^{N}_FusionRed_Cry2Olig_T_{ACT1} | |
| yNS50 | CEN.PK2-1C | Described herein |
| | YARCdelta5::P_{ADH1}_FusionRed_Cry2Olig_T_{ACT1} | |
| yNS50BY | BY4741 YARCdelta5::P_{ADH1}_FusionRed_Cry2Olig_T_{ACT1} | Described herein |
| yNS51 | yNS46 + pNS7 | Described herein |
| yNS52 | yNS21 + pNS8 | Described herein |
| yNS53 | yNS21 + pNS9 | Described herein |
| yNS54 | yNS52 | Described herein |
| | YARCdelta5::P_{ADH1}_FUS^{N}_Citrine_Cry2Olig_VioC_T_{ACT1} | |
| yNS55 | yNS52 | Described herein |
| | YARCdelta5::P_{ADH1}_VioC_FUS^{N}_Citrine_Cry2Olig_T_{ACT1} | |
| yNS50 | yNS53 | Described herein |
| | YARCdelta5:: | |
| | P_{ADH1}_FUS^{N}_FusionRed_Cry2Olig_VioE_T_{ACT1} | |
| yNS57 | yNS53 | Described herein |
| | YARCdelta5:: | |
| | P_{ADH1}_VioE_FUS^{N}_FusionRed_Cry2Olig_T_{ACT1} | |
| YEZ53 | CEN.PK2-1C + EZ-L176 | Described herein |
| YEZ140 | CEN.PK2-1C HIS3_{cg} | ⁴ |
| YEZ231 | CEN.PK2-1C HIS3::P_{PGK1}_FUS_Citrine_PixE_T_{CYC1} | Described herein |
| YEZ231BY | BY4741 HIS3::P_{PGK1}_FUS_Citrine_PixE_T_{CYC1} | Described herein |
| YEZ232 | YEZ231 YARCdelta5::P_{ADH1}_FUS^{N}_FusionRed_PixD_T_{ACT1} | Described herein |
| YEZ232BY | YEZ231BY YARCdelta5:: | Described herein |
| | P_{ADH1}_FUS^{N}_FusionRed_PixD_T_{ACT1} | |
| YEZ234 | CEN.PK2-1C YARCdelta5:: | Described herein |
| | P_{ADH1}_FUS^{N}_FusionRed_PixD_T_{ACT1} | |
| YEZ250 | YNS21 YARCdelta5::P_{ADH1}_Citrine_Cry2Olig_VioC_T_{ACT1}; | Described herein |
| | YARCdelta5::P_{ADH1}_FusionRed_Cry2Olig_VioE_T_{ACT1} | |
| YEZ255 | YEZ282 HIS3::P_{PGK1}_FUS^{N}_Citrine_PixE_VioE_T_{CYC1} | Described herein |
| YEZ257 | YEZ255 | Described herein |
| | YARCdelta5::P_{ADH1}_FUS^{N}_FusionRed_PixD_VioC_T_{ACT1} | |
| YEZ281 | YEZ255 YARCdelta5::P_{ADH1}_FUS^{N}_FusionRed_PixD_T_{ACT1} | Described herein |
| YEZ282 | BY4741 LEU2::P_{TEF1}_VioB_T_{ACT1} + P_{PGK1}_VioA_T_{CYC1} | Described herein |
| YEZ511 | YEZ257 + EZ-L859 | Described herein |
| YEZ512 | YEZ281 + pNS7 | Described herein |
| YEZ553 | YEZ255 YARCdelta5::P_{ADH1}_sFUS_PixD_VioC_T_{ACT1} | Described herein |
| YEZ554 | YEZ255 YARCdelta5::P_{ADH1}_sFUS_FusionRed_PixD_T_{ACT1} | Described herein |
| YEZ555 | YEZ231 YARCdelta5::P_{ADH1}_sFUS_FusionRed_PixD_T_{ACT1} | Described herein |
| MZW342 | S288C MATa/α HIS3::P_{Z3}_VioA; LEU2::P_{Z3}_VioB; | Described herein |
| | LYS2::P_{Z3}_VioE MET15::P_{Z3}_VioC; URA3::P_{Z3}_VioD; | |
| | CAN1::P_{ACT1}_yZ₃EV¹ | |
| MZW375 | S288C MATa/α HIS3::P_{Z3}_VioA LEU2::P_{Z3}_VioB | Described herein |
| | LYS2::P_{Z3}_VioE_μNSCCy MET15:: P_{Z3}_VioC_μNSCCy | |
| | URA3::P_{Z3}_VioD CAN1::P_{ACT1}_yZ₃EV¹ | |
| MZW377 | S288C MATa/α HIS3::P_{Z3}_VioA LEU2::P_{Z3}_VioB | Described herein |
| | LYS2::P_{Z3}_VioE_μNSCCy MET15:: P_{Z3}_μNSCCy_VioC | |
| | URA3::P_{Z3}_VioD CAN1::P_{ACT1}_yZ₃EV¹ | |
| MZW378 | S288C MATa/α HIS3::P_{Z3}_VioA LEU2::P_{Z3}_VioB | Described herein |
| | LYS2::P_{Z3}_ELK16_VioE MET15:: P_{Z3}_VioC_ELK16 | |
| | URA3::P_{Z3}_VioD CAN1::P_{ACT1}_yZ₃EV¹ | |

Yeast Strains and Transformations

Strain construction and transformations were performed using methods known in the art. For zeocin selection assays, DNA added ranged between 10μg-2 mg dependent on the target zeocin concentration.

Fluorescence Microscopy

Yeast strains were cultured overnight in a 24 well plate covered with foil. SC media was used to avoid the high auto-fluorescence of YPD. The following day, cultures were diluted 1:20 and allowed to grow for 2 hours such that the cells were in exponential phase. Wells of the microscopy plate were coated using 1 mg/mL Concanavalin A (Sigma) dissolved in 20 mM sodium acetate. After washing wells with ddH2O, yeast cultures were transferred and spun down at 1000 rpm for 3 min. All imaging was carried out using a 60X oil immersion objective (NA 1.4) on a Nikon A1 laser scanning confocal microscope. The laser was used at two different wavelengths: 488 nm for activation of Cry2 and 551 nm for visualization of FusionRed. Because 488 nm overlaps with the wavelength required for visualization of Citrine, 488 am was used as both the visualization and activation wavelength for Citrine-based constructs. Laser settings were 40% intensity for 488 nm and 30% intensity for 561 nm. Exposure times for both wavelengths was 200 ms.

Light Panel Set Ups

All light experiments were performed with blue LED panels (HARP New Square 12″ Grow Light Blue 517 LED 14 W), placed 40 cm from cell cultures. At these heights, the light panel outputs ranged from 73μmoles /m²/s to 82μmoles/m²s, based on measurements taken using a quantum meter (Model MQ-510 from Apogee Instruments). We selected for the light panels that omitted light with this range of intensities and did not use any light panels outside of this range.

Yeast Fermentation

Colonies from transformation plates were screened for DV and PDV production (8 colonies for each FUS^{N}-Cry2olig transformation and 8 colonies for each zeocin level for the PIXELL transformations). Colonies were used to inoculate 1 mL of SC-his+2% glucose media in 24-well plates and grown overnight at 30° C., 200 RPM, and under ambient conditions. Each culture was then diluted into 2 different plates and grown for 20 hours (one grown under blue light and the other wrapped in tinfoil and grown in the dark). Each colony was saved by plating onto an agar plate. Each culture was then spun down at 1,000 rpm and resuspended in fresh SC-his+2% glucose media. The plates were then grown under their respective light conditions for 96 hours before extraction and quantification of products. Colonies from the transformation plates of yeast containing the dual phase separation systems were screened (16 colonies each) using the same method but with SC-ura+2% glucose media. After selection for the best fold-change dependent on light condition, replicates of the best colonies were performed using the same protocol.

Extraction and Quantification of Violacein Pathway Products

1 mL of culture was transferred to a microcentrifuge tube and boiled at 95C for 15 minutes, vortexing halfway through. Cells were pelleted at 13000 rpm for 5 min and −800μL of supernatant was transferred to a new microcentrifuge tube. The new microcentrifuge tube was again pelleted at the same conditions and transferred to vials for analysis. Filtration of extracts were avoided because the Violacein pathway products were trapped by the filter membrane.

Extracts were run on an Alltech Alltima C18 column (250×4.6 mm, 5μm particle size) on an Agilent 1200 Series LC system with the following method (Solvent A is 0. 1% trifluoroacetic acid in acetonitrile; Solvent B is 0.1% trifluoroacetic acid in water): start at 5% A; from 0-10 min, 5%-95% A; from 10-13 min, hold at 95% A; from 13-13.5 min., 95%-5% A. The flow rate was 0.9 mL/min and products were monitored with an Agilent diode array detector (DAD) at 565 nm.

Product identities were confirmed using an Agilent 6120 Quadrupole mass spectrometer, using electrospray ionization in positive mode. Retention times were 10.04 min for proviolacein (m/z [M+H]+ of 328), 10.84 min for prodeoxyviolacein (m/z [M+H]+312), 10.95 min for violacein (m/z [M+H]+ of 344), and 12.25 min for deoxyviolacein (m/z [M+H]+ of 328).

Dual Clustering Can Result in Further Redirection of Metabolic Flux.

Simultaneous control over both a required endogenous pathway and a pathway towards the target product can increase product yields while maintaining healthy growth phases of culture. Using orthogonal systems for the formation of synthetic organelles can allow for rapid, controlled shifting of metabolic flux between competing branches of a metabolic pathway.

To implement this system, we tagged VioE and VioC with PIXELL and VioE and Viol) with Cry2. If VioDp is added to the system, flux is diverted towards the lower branches of the violacein pathway where VioDp converts PTDV to protoviolacein (PTV). In the absence of further enzymatic processing, PTV is spontaneously converted to proviolacein (PV). However, reaction of VioCp and PTV results in violacein. Starting from YEZ282, we integrated FUS-Citrine-Cry2-PixE-VioE (EZ-L557) for strain YEZ291. We then transformed with FUS-Citrine-Cry2-PixD-VioC (EZ-L527) and selected on 1,200 mg/L zeocin to make strain YEZ359, Finally, we added FUS-BFP-Cry2-VioD (EZ-L591) to make strain YEZ381. In this strain, Cry2-mediated blue light organelle formation shuttles PTDV flux towards PV and violacein while PIXELL-mediated dark organelle formation shuttles PTDV flux towards DV. The light induced fold change of violacein is larger than that of PV, predictably because in the dark, not only is the lower branch of the pathway not enhanced by clustering, but also VioCp, which is required to make violacein, is localized in PIXELL clusters. Control strains with non-tagged VioDp or VioCp and Cry2 or PIXELL tagged VioEp show no flux change from light to dark indicating that the change in product formation must be due to with substrate shuttling and not simply organelle formation. The described approach has applicability to anyone that does metabolic engineering including those seeking to employ enzyme clustering to enhance metabolic flux (by regulating the clustering state using light as described herein), and can be commercialized to generalize to any chemical with selectivity issues (particularly drug targets).

Thus, specific constructs and methods which can be used for, e.g., rapid and reversible clustering of proteins, have been described. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A synthetic protein comprising at least one inducible domain attached to a metabolic enzyme, wherein the inducible domain changes conformation in a manner that leads to oligomerization of the protein.

2. The synthetic protein according to claim 1, wherein a plurality of the synthetic proteins are inducibly clustered together.

3. The synthetic protein clusters according to claim 2, wherein a plurality of metabolic enzymes are attached to inducible domains and inducibly co-clustered together.

4. The synthetic protein according to claim 3, wherein a first inducible domain of the at least two different inducible domains is adapted for inducing clustering under a first condition, and a second inducible clustering tag of the at least two different inducible domains is adapted for inducing clustering in the absence of the first condition.

5. The synthetic protein according to claim 1, wherein the inducible domain is responsive to light having a wavelength in the visible light range.

6. The synthetic protein according to claim 1, wherein the inducible domain is responsive to the presence of a chemical or a change in pH.

7. The synthetic protein according to claim 1, wherein at least one inducible domain is further attached to an additional domain.

8. The synthetic protein according to claim 7, wherein the additional domain is an intrinsically disordered domain.

9. The synthetic protein according to claim 1, wherein the synthetic protein is encoded by a gene sequence wherein expression of the inducible domain is driven by a constitutive promoter.

10. The synthetic protein according to claim 1, wherein the synthetic protein is encoded by a gene sequence wherein expression of the inducible domain is under an inducible promoter.

11. A synthetic organism, comprising a synthetic protein according to claim 1.

12. The synthetic organism according to claim 11, wherein the synthetic organism is a strain of yeast, bacteria, mold, alga, plant, or a mammalian cell.

13. A method of controlling metabolic flux in a synthetic organism, comprising the steps of:
providing a synthetic organism according to claim 12; and
controlling the metabolic flux by exposing the synthetic organism to a first condition at a first point in time.

14. The method according to claim 13, wherein the first condition is light having a wavelength in the visible, UV, or infrared range.

15. The method according to claim 13, wherein the first condition is either the presence of a chemical or a temperature in within a first temperature range.

16. The method according to claim 13, further comprising modifying the metabolic flux by exposing the synthetic organism to a second condition at a second point in time.

17. The method according to claim 16, wherein exposing the synthetic organism to a second condition includes stopping the exposure of the synthetic organism to the first condition.

18. The method according to claim 17, wherein the synthetic organism is either first exposed to light and then exposed to dark, or first exposed to dark and then exposed to light.

19. The method according to claim 16, wherein exposing the synthetic organism to a condition recruits enzymes to a synthetic organelle.

20. The method according to claim 19, wherein exposing the synthetic organism to a second condition directs enzymes from a first synthetic organelle to a second synthetic organelle.

21. The method according to claim 19, wherein at least one of the pH or hydrophobicity of the synthetic organelle is modified by exposing the synthetic organism to a second condition that recruits a protein.

22. The method according to claim 19, wherein exposing the synthetic organism to a second condition recruits at least one material selected from the group consisting of a cofactor or a substrate.

23. The synthetic protein according to claim 1, further comprising an intrinsically disordered protein region (IDR) and at least one fluorescent protein attached to the at least one inducible domain.

24. The synthetic protein according to claim 23, wherein the IDR is at least a portion of FUS.

25. The synthetic protein according to claim 1, wherein the inducible domain is selected from the group consisting of Cry2, Cry2olig, PixE and PixD.
